(19) United States
(12) Patent Application Publication
(10) Pub. No.: US 2024/0123013 A1
(43) Pub. Date: Apr. 18, 2024

(54) SUPPLEMENT FOR ARTHRITIS AND PSORIASIS

(71) Applicant: CS Medica A/S, Kobenhavn O (DK)

(72) Inventors: Lone Henriksen, Greve (DK); Rosemarie Dauer, Hallerndorf (DE); Maria Agustina Duguine, Caba (AR)

(73) Assignee: CS Medica A/S, Kobenhavn O (DK)

(21) Appl. No.: 18/275,597

(22) PCT Filed: Feb. 7, 2022
(86) PCT No.: PCT/EP2022/052873

§ 371 Date: Aug. 2, 2023

(30) Foreign Application Priority Data
Feb. 5, 2021 (DK) PA 2021 70056

(51) Int. Cl. A61K 36/324; A61K 9/00; A61K 9/06; A61K 9/48; A61K 31/00; A61K 31/375; A61K 31/445; A61K 31/593; A61K 36/9066; A61P 17/06; A61P 19/02

(52) U.S. Cl. CPC A61K 36/324; A61K 9/0014; A61K 9/0056; A61K 9/06; A61K 9/4866; A61K 31/375; A61K 31/445; A61K 31/593; A61K 31/658; A61K 36/9066; A61P 17/06; A61P 19/02

(57) ABSTRACT

The present invention relates to an oral composition for use in the treatment and/or alleviation of symptoms such as pain and/or discomfort in a subject, such as pain or discomfort related to arthritis, or psoriasis. Such a treatment may further comprise application of a cannabinoid-comprising topical composition, such a CBD-comprising composition, such as a CBD-comprising hydroalcoholic gels. Different compositions comprising 3-O-Acetyl-11-Keto Beta Boswellic Acid, Curcuminoid, Vitamin C, and Vitamin D are disclosed.

### FIELD OF THE INVENTION

The present invention relates to a supplement for use in the treatment and/or alleviation of symptoms such as pain and/or discomfort in a subject, such as pain or discomfort related to arthritis, or psoriasis. Such a treatment may further comprise application of a cannabinoid-comprising topical composition, such a CBD-comprising composition, such as a CBD-comprising hydroalcoholic gel.

### BACKGROUND OF THE INVENTION

WO2020044123 relates to therapeutic combinations of Boswellia extract and cannabinoids.

WO2021023351 concerns CBD-comprising hydroalcoholic gels for treatment of arthritis and psoriasis.

WO2020024056 concerns compositions comprising cannabinoids and absorbable material and uses thereof.

US20100273895 pertains to formulations of cannabidiol and prodrugs of cannabidiol and methods of using the same.

KR102018221 concerns herbal extract-based compositions for preventing and alleviating arthritis, comprising extracts of boswellia, grape seeds, Curcuma longa L., green tea and ginger.

US2016/0129056 pertains to dietary supplements comprising Lactobacillus rhamnosus as well as further ingredients, such as e.g. ginger, vitamin D, curcumin, and Boswellia extract.

WO2019/153046 concerns orally administrable formulations for promoting gastrointestinal health, the formulation including a plurality of plant-based polyphenol sources and/or one or more components or derivatives thereof together with one or more of a fibre source, a sweetening agent and/or a flavouring agent

US2018/0289735 pertains to orally administrable formulations for alleviating joint pain and symptoms of osteoarthritis and/or rheumatoid arthritis comprising krill oil and/or marine oil in combination with other active constituents, such as astaxanthin, polymeric hyaluronic acid or sodium hyaluronate.

### SUMMARY OF THE INVENTION

Surprisingly and unexpectedly, the inventors have found that a composition comprising 3-O-Acetyl-11-Keto Beta Boswellic Acid (AKBBA), Curcuminoid(s), L-Ascorbic acid (Vitamin C), Cholecalciferol (Vitamin D), and optionally Piperidine, and/or Hesperidin preferably formulated for oral consumption, such as a supplement, has a beneficial impact on symptoms and/or discomfort related to a variety of conditions, comprising arthritis and/or psoriasis. Said beneficial effect can even be more pronounced, when said composition is used in combination with a topical composition for treatment of said condition(s), preferably a cannabinoid-comprising composition, such as a cannabidiol (CBD)-comprising composition.

The present invention comprises the following aspects:

In a first aspect, the present invention concerns a composition comprising 3-O-Acetyl-11-Keto Beta Boswellic Acid (AKBBA); Curcuminoid(s); L-Ascorbic acid (Vitamin C); Cholecalciferol (Vitamin D); and optionally Piperidine; and/or Hesperidin. Suitable compositions may comprise:
a. 1-15, 1-10, or 1.5-5% by weight 3-O-Acetyl-11-Keto Beta Boswellic Acid (AKBBA);
b. 1-20, 2-15, or 3-10% by weight Curcuminoid(s);
c. 1-20, 1-10, or 2-8% by weight L-Ascorbic acid (Vitamin C);
d. 0.00001-2, 0.0001-1.0, 0.0002-0.02% by weight Cholecalciferol (Vitamin D);
e. 0.01-2.0, 0.1-1.5, or 0.5-1.0% by weight Piperidine; and/or
f. 1-20, 1.5-10, or 2.5-3.0% by weight Hesperidin.

In some embodiments said composition can be formulated as a supplement, such as a capsule.

Arthritis supplements may generally comprise piperidine, while psoriasis supplements may often comprise hesperidin.

In a second aspect, the present invention relates to one or more compositions according to the first aspect for use as a medicament and/or in the in the treatment and/or alleviation of symptoms such as pain and/or discomfort in a subject, such as pain or discomfort related to a disease and/or condition, comprising autoimmune-related diseases, stress related conditions, arthritis, psoriatic arthritis and/or neurological pain, such as pain resulting from sclerosis, psoriasis (e.g. in particular red, dry, itchy, and/or scaly skin) psoriasis (e.g. in particular red, dry, itchy, and/or scaly skin), multiple sclerosis., herpes, such as herpes zoster, eczema, such as neurodermitis, Lichen simplex chronicus (LSC), atopic dermatitis (AD) aka atopic eczema, Lupus, such as systemic lupus erythematosus (SLE), scleroderma, urticaria, a painful condition associated with one or more hot and/or swollen joints, and/or a skin-, nerve-, joint condition.

In a third aspect, the present invention pertains to one or more composition according to the first or second aspect, wherein said composition is provided orally in combination with a cannabinoid-comprising composition for similar use formulated as a topical composition.

In some embodiments, said topical composition comprises the cannabinoid cannabinol (CBD). Said composition(s) can be e.g. be formulated as a hydroalcoholic gel. In some embodiments, said hydroalcoholic gel comprises:
i. cannabidiol (CBD) present in an amount of 0.1-20%, such as 0.1-10% or 0.2-5% (by weight);
ii. a skin penetration enhancer, such as a skin penetration enhancer present in an amount of 0.25-5%, or 0.5-2.5% (by weight);
iii. a low molecular weight alcohol, such as ethanol, such as a low molecular weight alcohol present in an amount of 5-50%, or 10-30% (by weight);
iv. one or more thickeners or gelling agents, such as one or more thickeners or gelling agents present in a total amount of 0.2-5%, or 0.4-2% (by weight); and
v. water in a quantity for the composition to a total of 100%.

In some embodiments the CBD used in the provision of the topical composition is crystalline. In some embodiments, said CBD is provided as—or capable of forming—needle-like crystals.

In a fourth aspect, the present invention concerns a method of treatment and/or alleviation of symptoms such as pain and/or discomfort in a subject, such as pain or discomfort related to a disease and/or condition, comprising autoimmune-related diseases, stress related conditions, such as one or more of: arthritis, rheumatoid arthritis, osteoarthritis, juvenile rheumatoid arthritis, psoriatic arthritis and/or neurological pain, such as pain resulting from sclerosis, psoriasis (e.g. in particular red, dry, itchy, and/or scaly skin), multiple sclerosis (MS), herpes, such as herpes zoster, seborrheic dermatitis, eczema, neurodermitis, Lichen simplex chronicus (LSC), atopic dermatitis (AD) aka atopic eczema, Lupus, such as systemic lupus erythematosus (SLE), scleroderma, urticaria, a painful condition associated with one or more hot and/or swollen joints, and/or a skin-, nerve-, joint condition.

Usually said method(s) comprise the use of a supplement according to any one of the preceding claims.

In some embodiments, said treatment comprises the use of a topical composition comprising a cannabinoid, such as cannabinol (CBD). Said composition(s) can be e.g. be formulated as a hydroalcoholic gel, such as an arthritis CBD gel, or a psoriasis CBD gel, e.g. according to the third aspect.

In some embodiments the CBD used in the provision of the topical composition is crystalline. In some embodiments, said CBD is provided as—or capable of forming—needle-like crystals. This may apply for all CBD-comprising topical compositions disclosed herein, in particular, but not exclusively for hydroalcoholic gel(s), such as an arthritis CBD gel, or a psoriasis CBD gel according to the third aspect.

In a fifth aspect, the present invention relates to a receptacle comprising a composition according to any one of the preceding aspects.

In a sixth aspect, the present invention pertains to a kit comprising a receptacle according to the fifth aspect, an instruction for use, and optionally a packaging.

### DESCRIPTION OF THE DRAWINGS/FIGURES

FIG. 1: microscope picture of cannabinol (CBD) forming needle-like crystals. The CBD crystals were sourced from www.enecta.com.

FIG. 2: microscope picture of cannabinol (CBD) forming cluster- or bunch-like crystals. The CBD crystals were sourced from www.gharma-hem.com.

### DETAILED DESCRIPTION OF THE INVENTION

### Definitions

In the context of the present invention, the singular form of a word may include the plural, and vice versa, unless the context clearly dictates otherwise. Thus, the references “a,” “an” and “the” are generally inclusive of the plurals of the respective terms. For example, reference to “an ingredient” or “a method” may include a plurality of such “ingredients” or “methods.”

Similarly, the words “comprise,” “comprises,” and “comprising” are to be interpreted inclusively rather than exclusively. Embodiments provided by the present disclosure may lack any element that is not specifically disclosed herein. Thus, a disclosure of an embodiment defined using the term “comprising” is also a disclosure of embodiments “consisting essentially of” and “consisting of the disclosed components”. Thus, the term “comprising” is generally to be interpreted as specifying the presence of the stated parts, steps, features, or components, but does not exclude the presence of one or more additional parts, steps, features, or components. For example, a composition comprising a chemical compound may thus comprise additional chemical compounds.

Where used herein, terms like “for example”, “e.g.” or “such as”, particularly when followed by a listing of terms, is merely exemplary and illustrative, and should not be deemed to be exclusive or comprehensive. Any embodiment disclosed herein may be combined with any other embodiment disclosed herein.

Unless expressed otherwise, all percentages expressed herein are by weight of the total weight of the composition. Thus, unless indicated otherwise, “%” indicates % weight/weight (w/w), also called weight % or by weight.

In the context of the present invention, the terms “about”, “around”, “approximately” or the symbol “˜” can be used interchangeably, and are meant to comprise variations and/or uncertainties generally accepted in the field, e.g. comprising analytical errors and the like. Thus “about” may also indicate measuring uncertainty commonly experienced in the art, which can be in the order of magnitude of e.g. +/−1, 2, 5, 10, or even 20 percent (%). Furthermore, “about” may be understood to refer to numbers in a range of numerals, for example the range of +/−20, +/−15, +/−10, +/−5, +/−2, +/−1, +/−0.5, +/−0.1% of the referenced number. Moreover, all numerical ranges herein should be understood to include all integers, whole or fractions, within the range.

As used herein, the term “in some embodiments” is meant to comprise both “in one embodiment” and “in some embodiments”.

In the context of the present invention, the terms “subject” or “patient” can be used interchangeably, and are meant to comprise a human, animal and/or mammal. In particular, a human subject can e.g. be selected from one or more of: female, male, senior, adult, adolescent, child, or infant. An animal subject can e.g. be selected from pet, husbandry, mammal, reptile, bird, and or animal in a Zoo.

In some embodiments, the subject or patient may suffer from one or more symptoms, conditions or diseases related to: autoimmune-related disease(s), stress related condition(s), arthritis; psoriasis, multiple sclerosis., herpes, herpes zoster, seborrheic dermatitis, eczema, neurodermitis, Lichen simplex chronicus (LSC), atopic dermatitis (AD) aka atopic eczema, Lupus, systemic lupus erythematosus (SLE), scleroderma, urticaria, and/or painful condition(s) associated with one or more hot and/or swollen joints.

As presented herein, surprisingly and/or unexpectedly, and from a long list of potential active component, the inventors have found that a composition for oral intake, such as supplement, can alleviate, lessen and/or improve such symptom(s), condition(s) or diseases in a subject, when only comprising a few relative and/or active ingredients.

In a first aspect, the present invention concerns a composition comprising 3-O-Acetyl-11-Keto Beta Boswellic Acid (AKBBA); Curcuminoid(s); L-Ascorbic acid (Vitamin C); Cholecalciferol (Vitamin D); and optionally Piperidine; and/or Hesperidin. Such compositions can be provided as supplement, such as dietary supplements.

In the following, the term “composition(s)” and “(dietary) supplement(s)” can be used interchangeably, unless clear from context.

In some embodiments, piperidine has been found to be advantageous for arthritis-, and/or similar conditions related conditions, while hesperidin has been found to be to be advantageous for psoriasis, and/or similar conditions, such as skin-related conditions.

It is generally advisable, to reduce the number and/or their concentration of components in such compositions to a minimum. Thereby, potential side effects side effects and/or in particular unforeseeable effects can be minimised. This can be in particular relevant, when such compositions are to be used on a daily basis for a prolonged period, such as over a period of several weeks or month.

Suitable concentration ranges, for compositions formulated as single, double, or triple oral doses disclosed herein comprise generally daily doses of around 0.5-1.0 or 0.5-2.0 g. Further suitable weights of such dosage forms are given below.

Unless clear from context, the above oral doses in terms of g/oral dosage form are generally applicable herein. It is submitted that the person skilled in the art will be able to provide suitable dosages, such as daily doses, based on the information given herein.

Generally, the required daily dose of a component, such as an active ingredient can be calculated based on a given formulation presented in % by weight herein, based on the weight of the oral dosage form, e.g. capsule, and the number of oral dosage forms per day: daily dose of an active ingredient=% by weight×weight of oral dosage form/100×number of oral dosage forms/capsules.

In some embodiments, a composition is provided comprising:
a. 1-15, 1-10, or 1.5-5% by weight 3-O-Acetyl-11-Keto Beta Boswellic Acid (AKBBA);
b. 1-20, 2-15, or 3-10% by weight Curcuminoid(s);
c. 1-20, 1-10, or 2-8% by weight L-Ascorbic acid (Vitamin C);
d. 0.00001-2, 0.0001-1.0, 0.0002-0.02% by weight Cholecalciferol (Vitamin D);
e. 0.01-2.0, 0.1-1.5, or 0.5-1.0% by weight Piperidine; and/or
f. 1-20, 1.5-10, or 2.5-3.0% by weight Hesperidin.

In some embodiments, the composition comprises (e) Piperidine, (f) Hesperidin, or (e)+Piperidine and (f) Hesperidin. In some embodiments, the composition comprises neither piperidine nor hesperidin.

In some embodiments, a composition for use in a treatment related to arthritis will comprise piperidine. In some embodiments, a composition for use in a treatment related to psoriasis will comprise hesperidin. In some embodiments, an arthritis or psoriasis composition may comprise both piperidine and hesperidin.

In some embodiments, a composition comprises:
a. 1-15% by weight 3-O-Acetyl-11-Keto Beta Boswellic Acid (AKBBA);
b. 1-20% by weight Curcuminoid(s);
c. 1-20% by weight L-Ascorbic acid (Vitamin C);
d. 0.00001-1.0% by weight Cholecalciferol (Vitamin D);
e. 0.1-1.5% by weight Piperidine; and/or
f. 1.5-10% by weight Hesperidin.

In some embodiments, a composition comprises:
a. 1.5-5% by weight 3-O-Acetyl-11-Keto Beta Boswellic Acid (AKBBA);
b. 3-10% by weight Curcuminoid(s);
c. 2-8% by weight L-Ascorbic acid (Vitamin C);
d. 0.0002-0.02% by weight Cholecalciferol (Vitamin D);
e. 0.5-1.0% by weight Piperidine; and/or
f. 2.5-3% by weight Hesperidin.

In some embodiments, a composition comprises 3-O-Acetyl-11-Keto Beta Boswellic Acid (AKBBA); Curcuminoid(s); L-Ascorbic acid (Vitamin C); Cholecalciferol (Vitamin D); and optionally Piperidine; and/or Hesperidin, wherein at least 3, 4, or all 5 of said components are provided in the following concentrations:
a. 1-15, 1-10, or 1.5-5% by weight 3-O-Acetyl-11-Keto Beta Boswellic Acid (AKBBA);
b. 1-20, 2-15, or 3-10% by weight Curcuminoid(s);
c. 1-20, 1-10, or 2-8% by weight L-Ascorbic acid (Vitamin C);
d. 0.00001-2, 0.0001-1.0, 0.0002-0.02% by weight Cholecalciferol (Vitamin D);
e. 0.01-2.0, 0.1-1.5, or 0.5-1.0% by weight Piperidine; and/or
f. 1-20, 1.5-10, or 2.5-3.0% by weight Hesperidin.

For applications related to arthritis, a suitable composition (also called “arthritis composition” or “arthritis supplement”) may e.g. comprise one or more of:
a. 1-15, 2-10, 3-5, or around 4% by weight 3-O-Acetyl-11-Keto Beta Boswellic Acid (AKBBA);
b. 2-20, 4-15, 6-10, or around 8% by weight Curcuminoid(s);
c. 1-20, 3-15, 4-8, or around 6% by weight L-Ascorbic acid (Vitamin C);
d. 0.00001-2, 0.0001-1.0, 0.00025-0.001, or around 0.00035% by weight Cholecalciferol (Vitamin D); and
e. 0.01-2.0, 0.1-1.5, 0.5-1.0, or around 0.7% by weight Piperidine.

In some embodiments, an “arthritis composition” can also be suitable for treatment of another condition or disease, such as one or more conditions or diseases disclosed herein, e.g. in the third or fourth aspect.

In some embodiments, an arthritis composition comprises components a-e, wherein at least 3, 4 or all 5 components are provided in a concentration as disclosed above.

In some embodiments, said arthritis composition may comprise one or more ingredients in a concentration as disclosed below:
a. around 4% by weight 3-O-Acetyl-11-Keto Beta Boswellic Acid (AKBBA);
b. around 8% by weight Curcuminoid(s);
c. around 6% by weight L-Ascorbic acid (Vitamin C);
d. around 0.00035% by weight Cholecalciferol (Vitamin D); and
e. around 0.7% by weight Piperidine.

In some embodiments, at least 3, 4 or all components a-e are provided in a concentration as disclosed above.

An arthritis composition may comprise further ingredients, such as Bromelain; and/or Collagen.

In some embodiment, an arthritis composition may further comprise:
m. 2-20, 4-15, 7-12, or around 9% by weight Bromelain (1200 GDU/g); and/or
n. 0.5-5, 0.7-2, 1.1-1.8, or around 1.4% by weight Collagen.

In some embodiments, a composition comprises at least 3, 4, 5, or 6 of ingredients a-e, m, and n. These components are believed to be useful for conditions related to pain and/or inflammation, such as arthritis.

In some embodiments, an arthritis composition can be formulated as a supplement comprising:
2-10, 3-5, or around 4% by weight 3-O-Acetyl-11-Keto Beta Boswellic Acid (AKBBA);
4-15, 6-10, or around 8% by weight Curcuminoid(s);
3-15, 4-8, or around 6% by weight L-Ascorbic acid (Vitamin C);
0.0001-1.0, 0.00025-0.001, or around 0.00035% by weight Cholecalciferol (Vitamin D);
0.1-1.5, 0.5-1.0, or around 0.7% by weight Piperidine;
4-15, 7-12, or around 9% by weight Bromelain (1200 GDU/g); and
0.7-2, 1.1-1.8, or around 1.4% by weight Collagen.

Arthritis compositions such as those presented above appear balanced in terms of concentration of active ingredients, the intended effect, and their applicability for an intended, daily use e.g. as a supplement, such as a supplement formulated as capsule. In some embodiments, an arthritis composition is formulated as single oral dose, e.g. as one capsule of 0.5-1.0, or around 0.72 g per day. Examples of different suitable oral dosage forms and their weights are disclosed elsewhere.

E.g. for applications related to psoriasis, a suitable composition (also called “psoriasis composition” or “psoriasis supplement”) may e.g. comprise:
a. 0.5-15, 1-10, 1.5-2.5, or around 2% by weight 3-O-Acetyl-11-Keto Beta Boswellic Acid (AKBBA);
b. 1-20, 2-15, 3-6, or around 4% by weight Curcuminoid(s);
c. 1-20, 1.5-10, 2-4, or around 3% by weight L-Ascorbic acid (Vitamin C);
d. 0.00001-2, 0.001-1.0, 0.005-0.02, or around 0.01% by weight Cholecalciferol (Vitamin D); and/or
f. 1-20, 1.5-10, 2.5-3.0, or around 2.8% by weight Hesperidin.

In some embodiments, a “psoriasis composition” can also be suitable for treatment of another condition or disease, such as one or more conditions or diseases disclosed herein, e.g. in the third or fourth aspect.

In some embodiments, a composition, such as a psoriasis composition may comprise:
a. around 2% by weight 3-O-Acetyl-11-Keto Beta Boswellic Acid (AKBBA); or
b. around 4% by weight Curcuminoid(s);
c. around 3% by weight L-Ascorbic acid (Vitamin C);
d. around 0.01% by weight Cholecalciferol (Vitamin D); and
f. around 2.8% by weight Hesperidin.

In some embodiments, at least 3, 4 or all components a-f are provided in a concentration as disclosed above.

In some embodiments, a composition, such as a psoriasis composition disclosed above, may comprise one or more of the following further components/ingredients:
q. Rosavin;
r. Zinc, such s Zinc gluconate;
s. Vitamin B12;
t. Withanolides;
u. Vitamin A;
v. Selene, such as L-Selenomethionine;
w. L-cysteine, such as L-cysteine HCL Mono;
x. L-glutamine;
y. Omega 3 fatty acids, such as eicosapentaenoic acid (EPA) and/or docosahexaenoic acid (DHA); and/or
z. Probiotic bacteria.

In some embodiments, a composition comprises at least 3, 4, 5, 6, 7, 8, 9 of ingredients q-z. These components are believed to be useful for skin-related conditions, such as arthritis.

In some embodiments, one or more further ingredients are provided in one or more of the following concentrations and/concentration ranges:
q. 0.1-5, 0.15-2.0, 0.25-0.8, or around 0.5% by weight Rosavin;
r. 0.25-5, 0.5-2.0, 0.75-1.25, or around 1% by weight Zinc;
s. 0.001-2.0, 0.002-1.0, 0.025-0.01, or around 0.07% by weight Vitamin B12;
t. 0.01-2.0, 0.1-1.5, 0.25-0.75, or around 0.5% by weight Withanolides;
u. 001-2.0, 0.002-1.0, 0.025-0.01, or around 0.07% by weight Vitamin A;
v. 0.0005-2.0, 0.00075-1.0, 0.001-0.005, or around 0.002 or 0.003% by weight Selene;
w. 0.1-20, 2-15, 7-12, or around 8.3% by weight L-cystein and/or L-cysteine HCL Mono;
x. 0.1-20, 2-15, 6-12, or around 9.0% by weigh L-glutamine;
y. 0.1-40, 0.5-20, 1.2-2.5, or around 1.85% by weight Omega 3 fatty acids such as, or comprising EPA and/or DHA; and/or
z. 0.2-5.0, 0.3-4.0, 0.4-1.3, or around 0.8% by weight Probiotic bacteria (5×10¹⁰ CFU/g).

In some embodiments, a psoriasis composition is provided, e.g. formulated as a supplement, comprising:
0.5-15, 1-10, 1.5-2.5, or around 2% by weight 3-O-Acetyl-11-Keto Beta Boswellic Acid (AKBBA); or
1-20, 2-15, 3-6, or around 4% by weight Curcuminoid(s);
1-20, 1.5-10, 2-4, or around 3% by weight L-Ascorbic acid (Vitamin C);
0.00001-2, 0.001-1.0, 0.005-0.02, or around 0.01% by weight Cholecalciferol (Vitamin D);
1-20, 1.5-10, 2.5-3.0, or around 2.8% by weight Hesperidin.
0.1-5, 0.15-2.0, 0.25-0.8, or around 0.5% by weight Rosavin;
0.25-5, 0.5-2.0, 0.75-1.25, or around 1.0% by weight Zinc;
0.001-2.0, 0.002-1.0, 0.025-0.01, or around 0.07% by weight Vitamin B12;
0.01-2.0, 0.1-1.5, 0.25-0.75, or around 0.5% by weight Withanolides;
001-2.0, 0.002-1.0, 0.025-0.01, or around 0.07% by weight Vitamin A;
0.0005-2.0, 0.00075-1.0, 0.001-0.005, or around 0.002 or around 0.003% by weight Selene;
00.2-5.0, 0.3-4.0, 0.4-1.3, or around 0.8% by weight Probiotic bacteria (5×10¹⁰ CFU/g).

In some embodiments, a composition is provided comprising 6, 7, 8, 9, 10, 11, or all 12 components above, and/optionally, in a concentration as disclosed above.

In some embodiments, the composition is formulated as oral dosage form (e.g. capsule) wherein 1, 2 or 3 of said oral dosage forms (e.g. capsules of 720 mg/day) provide a daily required dose. In some embodiments, said composition is formulated such that 2 or 3 capsules per day provide the daily dose. In some embodiments, more than 3 capsules per day provide the daily dose.

In some embodiments, a psoriasis composition may further comprise significant amounts of L-glutamine and/or L-cysteine, such as L-cysteine HCl mono. In some embodiments, one or more of L-glutamine, L-cysteine, or L-cysteine HCL mono provides a further positive effect when used daily, such as in the context of a skin-related conditions, such as psoriasis.

In some embodiments, a psoriasis composition may further comprise significant amounts of unsaturated fatty acids, such as omega 3 fatty acids. These can e.g. be provided from fish oil, and preferably from microencapsulated fish oil. Such formulations are e.g. rich in eicosapentaenoic acid (EPA) and/or docosahexaenoic acid (DHA), but they may also comprise further organic acids. As with other components disclosed above, omega 3 fatty acids and/or fish oil may contribute to a further advantage when consumed daily, such as in the context of a skin-related disease or condition, such as psoriasis. In some embodiments, concentrations of omega 3 fatty acids concern both EPA and DHA, i.e. as the sum of EPA and DHA.

Thus, in some embodiments it may be desirable to distribute the required daily doses, e.g. formulated as such as capsules, over more than 1, 2, 3 or more doses. Thus in some embodiments, a glutamine and/or cysteine comprising psoriasis composition, such as a supplement, may comprise one or more of:
0.5-5, 0.7-4.0, 1.0-2.0, or around 1.4% by weight 3-O-Acetyl-11-Keto Beta Boswellic Acid (AKBBA); or
1-20, 2-10, 2-4, or around 2.8% by weight Curcuminoid(s);
1-20, 1.1-10, 1.5-4.0, or around 2% by weight L-Ascorbic acid (Vitamin C);
0.00001-2.0, 0.001-1.0, 0.005-0.02, or around 0.01% by weight Cholecalciferol (Vitamin D);
1-20, 1.2-10, 1.5-2.5, or around 1.85% by weight Hesperidin;
0.001-5.0, 0.15-2.0, 0.2-0.6, or around 0.35% by weight Rosavin;
0.25-5, 0.3.5-2.0, 0.5-1.0, or around 0.7% by weight Zinc;
0.001-2.0, 0.002-1.0, 0.025-0.01, or around 0.05% by weight Vitamin B12;
0.01-2.0, 0.1-1.0, 0.2-0.5, or around 0.3% by weight Withanolides;
001-2.0, 0.002-1.0, 0.025-0.75, or around 0.05% by weight Vitamin A;
0.0005-2.0, 0.00075-1.0, 0.001-0.005, or around 0.002 or 0.003% by weight Selene, and/or selenomethionine;
0.1-20, 2-15, 7-12, or around 8.3% by weight L-cysteine and/or L-cysteine HCL Mono;
0.1-20, 2-15, 6-12, or around 9.0% by weigh L-glutamine;
0.1-40, 0.5-20, 1.2-2.5, or around 1.85% by weight Omega 3 fatty acids, such as (EPA) and/or (DHA); and/or
0.2-5.0, 0.3-4.0, 0.4-1.3, or around 0.8% by weight Probiotic bacteria (5×10¹⁰ CFU/g).

In some embodiments, a composition is provided comprising 9, 10, 11, 12, 13, 14, or all 15 components above; and/optionally, in a concentration as disclosed above.

Psoriasis compositions such as those presented above appear balanced in terms of concentration of active ingredients, the intended effect, and their applicability for an intended, daily use e.g. as a supplement, such as a supplement formulated as capsule. In some embodiments, a psoriasis composition can be formulated as single oral dose, e.g. as one capsule of 0.5-1.0, or around 0.72 g per day. Sometimes, due to the number and amount of further ingredients, daily doses may comprise more than on oral dosage form/capsule, such as 2, 3, or more. Examples of different suitable oral dosage forms and their weights are disclosed elsewhere.

In some embodiments, the composition is formulated as oral dosage form (e.g. capsule) wherein 1, 2 or 3 of said oral dosage forms (e.g. capsules) of 720 mg/day. In some embodiments, 2 capsules per day will provide the required daily dose. In some embodiments, 3 capsules per day will provide the required daily dose. In some embodiments, more than 3 capsules per day will provide the require daily dose.

In some embodiments, the composition is formulated as an oral dosage form, such as capsule of around 0.5-1.0 or 0.72, wherein the daily dose for a subject, such as an adolescent, adult or senior is two or three oral dosage forms/day.

As disclosed above, in some embodiments, compositions, such as arthritis- and/or psoriasis compositions can be formulated for oral intake or consumption, formulated e.g. as a tablet, pill, or capsule. Such formulation(s) may comprise an acceptable excipient, such as 1-80, 2-70, 3-60, 4-50, 5-30, 8-20, 10-15, or around 12% by weight. An example of such an excipient is Rice Bran Extract. In some embodiments, said excipient may be present in a concentration of at least 5, 10, 20, 30, 40, 50, 60, 70, or 80% by weight, or more.

When formulated as a capsule, said composition may comprise a capsule shell, such as conventional capsule shell. This shell may e.g. comprise 4-50, 5-30, 10-25, 15-20, or around 17% by weight capsule shell. In some embodiments, said capsule shell may comprise a glazing agent, such as Hydroxypropyl Methylcellulose, and/or a colouring agent, such as TiO₂.

Generally, in the embodiments presented herein, the percentages are given for compositions which may comprise an excipient and/or a capsule shell. A person skilled in the art can derive other dosages, e.g. for an oral dosage form without said excipient and/or capsule shell without undue burden.

In some embodiments, the composition is formulated as a supplement for daily intake, such as 1-5, e.g. 1, 2 or 3 pills, tablets, capsules, or the like per day.

In some embodiments, the composition is formulated for oral intake, such as an oral dosage form formulated as tablet, pill, or capsule with a weight of around 0.1-2.0 g, 0.4-1.5 g, 0.5-1.0 g, or around 0.72 g. In some embodiments, said daily doses forms have a weight of around 0.5, 0.6, 0.7, 0.8, 0.9, 1.0, 1.2, 1.4, 1.6, 1.8, or 2.0 g. In some embodiments, said weight can be less than 0.5 g, 0.5-0.6, 0.6-0.7, 0.7-0.8, 0.8-0.9, 0.9-1.0 g, 1.0-2.0 g, or (iv) more than 2.0 g. In some embodiments, the supplements are formulated as capsules, with a weight of around 0.6-0.8 g, such as around, 0.6, 0.625, 0.65, 0.675, 0.7, 0.71, 0.72, 0.73, 0.74, 0.75, 0.8, 0.9 or 1.0 g.

The ingredients/components/constituents of a composition can be provided in different forms, ranging from essentially pure form to more complex extract.

Suitable sources of these components, such as when not using a pure, or essentially pure form of said components, may e.g. comprise one or more of the following: 3-O-Acetyl-11-Keto Beta Boswellic Acid (AKBBA) can be provided from Boswellia Serrata Resin Extract.

Curcuminoid(s) can be provided from Curcuma Longa Rhizomes Extract.

Vitamin C can be provided from Rosehip Fruit Extract (Rosa canina).

Vitamin D can be provided from algae, such as Vita-algae D®; Vita-algae D® 100,000iug powder is a light-yellow powder. It is a vegan source of vitamin D3 sourced 100% from algae. It can be a 100% Vegetable source of vitamin D3.

Piperine can be provided from Black Pepper Fruit Extract (Piper nigrum).

Hesperidin can be provided from Bitter Orange Peel Extract (Citrus aurantium).

Bromelain can be provided from Pineapple (Ananas comosus) (1200 GDU/g).

Collagen can be provided from Standardized Chicken Cartilage Powder [25% Total Collagen].

Rosavin can be provided from Rhodiola Rosea Root Extract (Rhodiola rosea) [3% Rosavin].

Zinc can be provided as Zinc Gluconate.

Vitamin B12 can be provided from Cyanocobalamin and/or Methylcobalamin.

Withanolides can be provided from Ashwagandha Root Extract (Withania somnifera) [7% Withanolides].

Vitamin A can be provided from Retinyl Acetate.

Selenium can be provided from L-Selenomethionine, such as from SeleniumSeLECT® (Sabinsa).

L-Cysteine can be provided from L-cysteine HCL mono.

Omega 3 fatty acids can be provided from microencapsulated fish oil comprising e.g. eicosapentaenoic acid (EPA) and/or docosahexaenoic acid (DHA), such as 10-80, or around 40 mg/g EPA and/or 20-200, or around 105 mg/g docosahexaenoic acid (DHA).

Probiotic bacteria can be provided from Bacillus coagulans ATCC7050 (5 10 CFU/g). In some embodiments, one or more different strains of a probiotic bacterium can be provided. In some embodiments, the daily oral dose of probiotic microorganism(s), such as bacillus, and/or lactic acid bacteria can be around 1×10¹⁰ CFU or more, between 1×10⁷ or 10⁶ and 1×10¹⁰ CFU, or around 1-6×10⁸ CFU, such as 2-5, or around 6×10⁸ CFU/day. In some embodiments, the daily dose can be lower than 1×10⁶. In some embodiments, the daily dose can be higher than 1×10¹⁰.

In some embodiments, AKBBA and/or Boswellia Serrata Resin Extract provides and/or contributes to pain relief.

In some embodiments, Curcuminoid(s) and/or Curcuma Longa Rhizomes Extract provides and/or contributes to an anti-inflammatory effect. In some embodiments, Curcuminoid(s) and/or Curcuma Longa Rhizomes Extract provides and/or contributes to an antioxidant effect.

In some embodiments, Vitamin C and/or Rosehip Fruit Extract provides and/or contributes to an antioxidant effect. In some embodiments, Vitamin C and/or Rosehip Fruit Extract provides and/or contributes to a normal collagen rebuild and/or normal bone function/composition.

In some embodiments, Vitamin D, such as Vitamin D3 and/or a Vita-algae composition provides and/or contributes to one or more of: skin healing, bone health, improvement of conditions related to psoriasis.

In some embodiments, Piperine and/or Black Pepper Fruit Extract provides and/or contributes to an improved bioavailability and/or an antioxidant effect.

In some embodiments, Hesperidin and/or Bitter Orange Peel Extract provides and/or contributes to an improved bioavailability and/or anti-inflammatory effect. Furthermore, Hesperidin and/or Bitter Orange Peel Extract may provide and/or contribute to a stabilizing effect, such as a stabilizing effect on vitamins.

In some embodiments, Bromelain provides and/or contributes to an anti-inflammatory effect.

In some embodiments, Collagen and/or Standardized Chicken Cartilage Powder provides and/or contributes to rebuilding collagen, such as collagen in joints.

In some embodiments, Rosavin and/or Rhodiola Rosea Root Extract provides and/or contributes to an improved and/or more balanced immune system.

In some embodiments, Zinc and/or Zinc Gluconate provides and/or contributes to an improved and/or faster healing of tissues. In some embodiments, Zinc and/or Zinc gluconate improves the production of oil(s) in/on of the skin and/or related tissue(s).

In some embodiments, Vitamin B12, Cyanocobalamin, and/or Methylcobalamin provides and/or contributes to an improved health of skin and/or hair.

In some embodiments, Withanolides and/or Ashwagandha Root Extract provides and/or contributes to a reduction of stress.

In some embodiments, Vitamin A and/or Retinyl Acetate provides and/or contributes to an improved skin healing process.

In some embodiments, Selene, e.g. provided as L-Selenomethionine and/or SeleniumSeLECT) provides and/or contributes to an antioxidant effect. In some embodiments, L-Selenomethionine and/or SeleniumSeLECT provides and/or contributes to reducing oxidative stress related to psoriasis.

In some embodiments, L-Cysteine and/or L-cysteine HCL mono provides and/or provides and/or contributes to one or more of: skin healing, improvement of conditions related to psoriasis.

In some embodiments, L-Glutamine provides and/or contributes to an anti-inflammatory effect. In some embodiments, L-Glutamine provides and/or contributes to an anti-autoimmune illness effect, such as reduction of the severity of an autoimmune condition.

In some embodiments, Omega 3 fatty acids, fish oil, and/or eicosapentaenoic acid (EPA) and/or docosahexaenoic acid (DHA) provide and/or contribute to one or more of: support and or improvement of immune function, prevention of inflammatory diseases; regulating the skin's oil production and/or balance; improved balanced hydration; softening rough/dry skin, and/or have a soothing effect on skin irritation and/or dermatitis; including any combination thereof.

In some embodiments, probiotic bacteria such as Bacillus coagulans ATCC7050 provides and/or contributes to one or more of: better digestion, removal of toxins from body, reduced stress level in the body, improved gut flora, improved skin flora.

In some embodiments, a composition as disclosed herein, in particular, but not exclusively, formulated as a supplement, does not comprise glucosamine.

In some embodiments a composition as disclosed herein, in particular, but not exclusively, formulated as a supplement, may comprise glucosamine in a concentration below 1, 0.5, or 0.1% by weight.

Generally, glucosamine may not be desired in the context of the present invention. Usually, high doses of glucosamine are needed, thus requiring large daily doses by weight, thus too large for a single, or two capsules per day, which is not ideal for a subject. Furthermore, it is believed that glucosamine is less efficient/effective than the oral compositions of the present invention. However, in some embodiments, glucosamine can be taken as additional supplement or ingredient, e.g. in the context of any of the treatments disclosed herein.

In some embodiments, the concentration of components as disclosed herein are daily doses, such as per oral dosage form per day, e.g. formulated as 1, 2, 3 or more capsules of 720 mg/day. In some embodiments, a single daily dose is preferred. In some embodiments, 2 or 3 daily doses are preferred. In some embodiments, an oral dosage form is formulated for more than 3 daily doses. In some embodiments, it can be advantageous to provide the daily doses in the form of 2 or 3 oral dosage forms/capsules, so smaller doses can be consumed e.g. morning, noon and evening, such as when eating breakfast, lunch or dinner, e.g. in order to reduce a negative impact of one or more of the components on the digestive system, and/or in order to provide a more uniform level of said components in the subject, thus avoiding larger fluctuations.

In a second aspect, the present invention relates to one or more compositions e.g. according to the first aspect for use as a medicament and/or in the in the treatment and/or alleviation of symptoms such as pain and/or discomfort in a subject, such as pain or discomfort related to a disease and/or condition, comprising autoimmune-related diseases, stress related conditions, arthritis, rheumatoid arthritis, osteoarthritis, juvenile rheumatoid arthritis, psoriatic arthritis and/or neurological pain, such as pain resulting from sclerosis, psoriasis (e.g. in particular red, dry, itchy, and/or scaly skin), multiple sclerosis, herpes, such as herpes zoster, seborrheic dermatitis, eczema, such as neurodermitis (Lichen simplex chronicus (LSC), atopic dermatitis (AD) aka atopic eczema, Lupus, such as systemic lupus erythematosus (SLE), scleroderma, urticaria, a painful condition associated with one or more hot and/or swollen joints, and/or a skin-, nerve-, joint condition.

In some embodiments, a composition according to the first aspect, such as an arthritis or psoriasis composition can be used as a medicament. In some embodiments, the composition can be used as a medicament and/or in the treatment of one or more of e.g.: autoimmune-related diseases, stress related conditions, arthritis, rheumatoid arthritis, osteoarthritis, juvenile rheumatoid arthritis, psoriatic arthritis and/or neurological pain, such as pain resulting from sclerosis, psoriasis (e.g. in particular red, dry, itchy, and/or scaly skin), multiple sclerosis, herpes, such as herpes zoster, seborrheic dermatitis, eczema, such as neurodermitis (Lichen simplex chronicus (LSC), atopic dermatitis (AD) aka atopic eczema, Lupus, such as systemic lupus erythematosus (SLE), scleroderma, urticaria, a painful condition associated with one or more hot and/or swollen joints, skin condition, nerve and/or joint condition; and/or a skin-, nerve-, joint condition.

In some embodiments, the subject or patient is a human, such as one or more of: female, male, senior, adult, adolescent, child, or infant. In some embodiments, the subject is an animal, such as a pet, e.g. dog or cat, husbandry (cow, horse, camel), including animals in a zoo. In some embodiments, the subject is a mammal.

In some embodiments, a composition according to the first embodiment can be used in the treatment and/or alleviation of symptoms of a skin, nerve and/or joint condition.

In some embodiments, a composition according to the first embodiment can be used in the treatment and/or alleviation of symptoms related to an autoimmune condition.

In some embodiments, a composition according to the first embodiment can be used in the treatment and/or alleviation of symptoms related to arthritis. E.g. arthritis compositions as disclosed herein are very suitable in this context.

In some embodiments, a composition according to the first embodiment can be used in the treatment and/or alleviation of symptoms related to psoriasis. E.g. psoriasis compositions as disclosed herein are very suitable in this context.

In a third aspect, the present invention pertains to one or more composition according to the first or second aspect, wherein said composition is provided orally in combination with a treatment comprising the use of a composition formulated as a topical composition.

In some embodiments, a composition, such as a supplement, is provided orally in combination with a treatment comprising the use of a cannabinoid-comprising composition formulated as a topical composition.

Thus in some embodiments, the topical composition does not comprise one or more cannabinoid. In some embodiments, said composition may comprise one or more cannaboid(s), such as any one of the cannabinoids disclosed herein, in particular below.

In some embodiments, said cannabinoid is CBD and/or comprises CBD in a physiological active amount, such as 0.01-5.0, 0.1-2.5, 0.5-2%, or 0.75-1.5%.

In some embodiments, said CBD-comprising composition is e.g. formulated as cream, ointment, salve, gel, skin-patch, spray, or any other form known in the art topical formulations.

In some embodiments, the topical composition is formulated as a hydroalcoholic gel. Such gels usually comprise 5-50%, or 10-30% by weight of one or more low molecular weight alcohol, e.g. a C1-C3 alcohol, such as EtOH.

In some embodiments, the hydroalcoholic gel is, is essentially, and/or comprises a composition as disclosed in any one of DK priority application PA 2019 70497, DK priority application PA 2020 70343, and/or WO2021023351, such as disclosed in any one of the claims of said PA 2019 70497, PA 2020 70343, and/or WO2021023351, or in any example(s) or embodiment(s) disclosed therein.

In some embodiments, such a hydroalcoholic gel may comprise one or more, or all of:
i. cannabidiol (CBD) present in an amount of 0.1-20%, such as 0.1-10% or 0.2-5% (by weight);
ii. a skin penetration enhancer, such as a skin penetration enhancer present in an amount of 0.25-5%, or 0.5-2.5% (by weight);
iii. a low molecular weight alcohol, such as ethanol, such as a low molecular weight alcohol present in an amount of 5-50%, or 10-30% (by weight);
iv. one or more thickeners or gelling agents, such as one or more thickeners or gelling agents present in a total amount of 0.2-5%, or 0.4-2% (by weight); and
v. water in a quantity for the composition to a total of 100%.

In some embodiments, a hydroalcoholic gel may further comprise sodium chloride, in particular see salt, or more preferred dead sea salt. In some embodiments, a hydroalcoholic gel comprises significant amounts of salt, such as 10-25% (by weight). Generally, salt-comprising gels are believed to be very useful for skin-related conditions, such as in particular, but not exclusively, psoriasis. In some embodiments, such a psoriasis hydroalcoholic gel does not comprise citric acid and/or citrate.

In some embodiments, a hydroalcoholic gel may comprise one or more skin care or skin hydrating/moisturizing agents, e.g. selected from barbadensis leaves, panthenol, retinyl palmitate, and/or glycerine, including any combination thereof. Suitable concentration ranges of said hydrating/moisturizing agents may e.g. comprise:
0.01-1.0% (by weight) extract of aloe barbadensis leaves,
1-5% (by weight) of panthenol,
0.1-1-5% (by weight) of retinyl palmitate, and
0.5-5% (by weight) of glycerine, including any combination(s) thereof.

In some embodiments, the cannabinoid, e.g. cannabidiol used in preparation of a topical composition, such as a hydroalcoholic gel, is provided in a crystalline or pure form. Generally, such a pure form comprises very little or no further cannabinoids; or in physiologically insignificant amounts. In some embodiments, the cannabinoid, such as CBD comprises e.g. less than 1.5%, 1.0% or 0.5% (by weight) of any one of: Cannabidivarin (CBDV), Cannabidiolic acid (CBDA), Cannabigerol (CBG), Cannabinol (CBN); and/or less than 1.0% of Tetrahydrocannabinol (THC).

In some embodiments, provision of the cannabinoid in pure form provides an advantage in terms of effect per dosage, such that a lower concentration of the pure or crystalline cannabinoid is needed in comparison to a conventional mixture comprising a variety of further cannabinoids, where a higher concentration of said cannabinoid is needed. Without wanting to be bound by any theory, this effect appears surprising, as the common belief is that a mixture of cannabinoids is better and/or more efficient than a single, pure cannabinoid in cannabinoid-comprising topical compositions.

Generally, for some hydroalcoholic gels, the presence of oils and/or fats, such as vegetable and/or mineral oil can be undesirable, e.g. with respect to efficiency of treatment. Thus, in some embodiments, the hydroalcoholic gel comprises less than 2.0, 1.0%, 0.5, or 0.1% (by weight) oil, such as vegetable and/or mineral oil. Without wanting to be bound by any theory, it is believed that the presence of oil reduces the efficiency of a cannabinoid, such as CBD, requiring higher concentrations or dosages.

In some embodiments, a hydroalcoholic gel may further comprises one or more further components selected from 0.5-5% (by weight) of menthol and/or 0.1-2% (by weight) of camphor and/or 0.5-1.5% (by weight) of eucalyptus oil.

In some embodiments, a hydroalcoholic gel may comprise one or more skin penetration enhancer. These can e.g. be selected from isopropyl myristate, dimethylsulfoxid (DMSO), urea, or any combinations thereof.

In some embodiments, a hydroalcoholic gel may comprise one or more thickener and/or gelling agent, such as thickener and/or gelling agent selected from acrylate cross polymers, hydroxyethyl cellulose, xanthan gum and/or any combinations thereof.

In some embodiments, a hydroalcoholic gel may comprise one or more pharmaceutically acceptable adjuvants selected from antioxidants, emulsifiers, pH regulating agents, such as acids or bases, buffers, stabilizers, colorants, and/or any combination thereof.

In some embodiments, a hydroalcoholic gel may be formulated as a cosmetic skin hydrating and/or skin care composition.

In some embodiments, the topical composition, such as the cannabinoid-(e.g. CBD) comprising composition is a composition for use as a medical composition in a local topical application in the treatment or alleviation of symptoms, in particular pain, resulting from arthritis, e.g. rheumatoid arthritis, osteoarthritis, juvenile rheumatoid arthritis and/or psoriatic arthritis and/or neurological pain, such as pain resulting from sclerosis, e.g. multiple sclerosis, in a subject.

In some embodiments, the topical composition is a composition for use as a medical composition in local a topical application in the treatment or alleviation of symptoms of psoriasis, in particular of red, dry, itchy, and/or scaly skin in a subject.

In some embodiments, the topical composition is applied topically to a local skin area of a subject 1-5, 1-3, or 1-2 times per day.

In some embodiments, the topical composition is applied topically to a skin area of a subject, in an amount of 5-100, or 10-50 mg/cm² per application.

In some embodiments, said a cannabinoid-comprising composition (e.g. CBD), does not comprise one or more further cannabinoid(s), such as one or more hallucinogenic and/or non-hallucinogenic cannabinoid, and/or wherein said further cannabinoid(s) is/are present in amount below 10, 5, 2, 1, 0.5% by weight in relation to the main cannabinoid, such as CBD.

In some embodiments, a cannabinoid-comprising composition may comprise more than one cannabinoids, such as one or more further cannabinoid(s), e.g. selected from one or more of: THC (tetrahydrocannabinol), THCA (tetrahydrocannabinolic acid), CBDA (cannabidiolic acid), CBN (cannabinol), CBG (cannabigerol), CBC (cannabichromene), CBL (cannabicyclol), CBV (cannabivarin), THCC (tetrahydrocannabiorcol), THCV (tetrahydrocannabivarin), THCP (tetrahydrocannabiphorol), CBDV (cannabidivarin), CBCV (cannabichromevarin), CBGV (cannabigerovarin), CBGM (cannabigerol monomethyl ether), CBE (cannabielsoin), CBT (cannabicitran), including one or more cannabinoids of the following types: CBG-type, CBC-type, CBD-type other than CBD, THC-type, CBN-type, CBE-type, iso-THC-type, CBL-type, CBT-type, including any combination(s) thereof.

In some embodiments, a further cannabinoid is provided in a ratio CBD:further cannabinoid of >10:1, 10:1-5:1, 5:1-2:1, 2:1-1:1, 1:1-1:2, 1:2-1:5, or <1:5. In some embodiments, said ratio are CBD:“sum of further cannabinoids”. Said ratios are usually by weight.

In some embodiments, a hydroalcoholic gel can be formulated in particular for treatment of arthritis-related conditions. Such a gel may comprise one or more of:
0.5-2.0% (by weight) or more preferred ˜1.0% (by weight) of cannabidiol;
10-30% (by weight) of ethanol or more preferred ˜25% (by weight) of ethanol, in particular 96% ethanol, such as 96% denatured ethanol;
0.5-5% (by weight) or more preferred ˜2% (by weight) menthol;
0.1-2% (by weight) or more preferred 0.55% (by weight) of camphor;
0.5-1.5% (by weight) or more preferred ˜0.9% skin penetration enhancer, such as isopropyl myristate;
0.4-2% (by weight) or more preferred ˜1% thickeners and/or gelling agents, such as acrylate crosspolymer;
0.05-0.5% (by weight) or more preferred ˜0.2% phytic acid (e.g. of 50% purity); and
water in a quantity for the composition to a total of 100% (by weight), such as 50-80, or around ˜68% (by weight), said water being preferably water of drinking water quality.

In some embodiments, a hydroalcoholic gel formulated in particular for treatment of arthritis-related conditions may comprise all, or at least 5, 6, 7 or 8 of the above components. Usually, said components will be provided in any of the concentrations and/or concentration ranges disclosed above.

In some embodiments, a hydroalcoholic gel can be formulated in particular for treatment of psoriasis-related conditions, said gel comprising:
0.5-2.0% (by weight) or more preferred ˜1.0% (by weight) cannabidiol;
0.3-1.5% (by weight) or more preferred ˜0.9% (by weight) skin penetration enhancer, in particular isopropyl myristate;
0.1-0.5% (by weight) or more preferred ˜0.2% (by weight) glycerine;
10-30% (by weight) or more preferred ˜15% (by weight) ethanol, in particular 96% ethanol, such as 96% denatured ethanol;
0.5-2.5% (by weight) or more preferred ˜1.5% (by weight) one or more thickeners or gelling agents, in particular hydroxyethyl cellulose;
10-20% (by weight) or more preferred ˜15.0% (by weight) sodium chloride, in particular sea salt, or more preferred Dead Sea salt;
% (by weight) or more preferred ˜0.05% (by weight) extract of aloe barba-densis leaves;
1.5-4.0% (by weight) or more preferred ˜2.75% (by weight) panthenol;
0.1-0.5% (by weight) or more preferred ˜0.3% (by weight) retinyl palmitate;
0.1-0.3% (by weight) or more preferred ˜0.2% (by weight) glycerine;
0.01-0.03% (by weight) or more preferred ˜0.02% (by weight) of an organic acid and/or salt thereof, e.g. lactic acid and/or acetic acid;
water in a quantity for the composition to a total of 100% (by weight), such as 50-80% (by weight), more preferred ˜61% (by weight), said water being preferably water of drinking water quality.

In some embodiments, a hydroalcoholic gel formulated in particular for treatment of arthritis-related conditions may comprise all, or at least 6, 7, 8, 9, 10, 11 of the above components. Usually, said components will be provided in any of the concentrations and/or concentration ranges disclosed above.

In some embodiments, the organic acid and/or 0.01-0.03% (by weight) or more preferred ˜0.02% (by weight) of an organic acid and/or salt thereof is not citric acid/citrate. Generally, it is believed that the presence of citric acid is not desired in compositions concerning skin-related conditions, such as psoriasis. Without wanting to be bound by any theory, it is believed that lactic acid/lactate and/or acetic acid/acetate are more suitable.

Concerning treatment of arthritis-related conditions, a combination of “arthritis supplement” and “arthritis topical composition” can be desirable.

Likewise, with respect to treatment of psoriasis-related conditions, a combination of “arthritis supplement” and “arthritis topical composition” can be desirable.

Concerning the CBD used in the preparation or formulation of a CBD-comprising topical formulation, in some embodiments, the CBD used in the provision of the topical composition is crystalline.

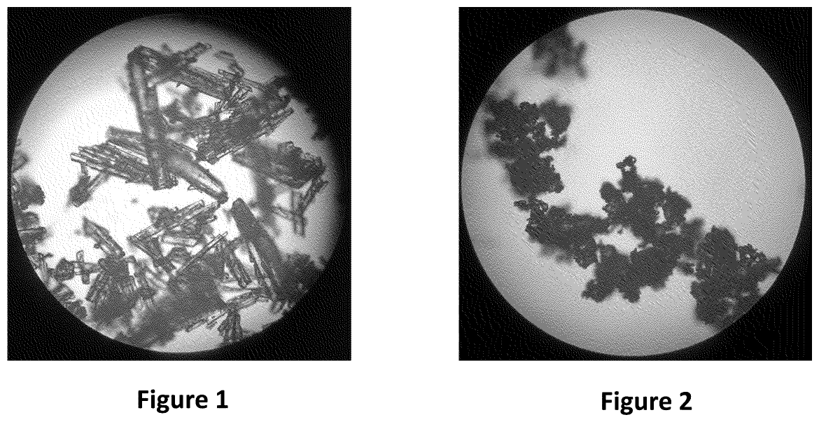

In some embodiments, the CBD used for providing a topical composition as disclosed above is characterized by a certain features, such as the crystal structure and/or conformation. It has been observed by the inventors, see e.g. Example 12, that CBD with a needle-like crystal structure (=crystal structure A; see FIG. 1), surprisingly and unexpectedly, appears significantly more potent than CBD with a different crystal structure, a non-needle like structure, also termed “bunch-like or “cluster-like” herein (=crystal structure B; see FIG. 2).

In some embodiments, the CBD possesses, when crystalline, or is capable of forming a needle-like crystal structure. In some embodiments, CBD of crystal structure A (or capable of forming needle-like crystals) is at least 1.5, 2, 3, 4, 5, 7.5, 10, 15 or 20 times more potent than CBD of crystal structure B (or capable of forming cluster/bunch-like crystals). It can be speculated if the CBD needs to be in an active form, such one or more specific conformation(s) in order to be active upon administration to a subject, such as in a topical formulation. Lack of activity or potency can also be caused by a lower uptake rate and/or difficulties in passing through the skin.

Without wanting to be bound by any theory, it is believed that the difference in crystal structure may be caused by a different molecular structure, such as a different conformation. This could e.g. be due to a failure of the subject's body to recognize the “wrong” CBD conformation or the like. It is conceivable that the differences in CBD crystal structure are caused by a different extraction process. In particular, the CBD disclosed in FIG. 1 was provided by an extraction process, comprising extraction with isopropanol, distillation and crystallization with heptane (see e.g. Example 12), while the CBD disclosed in FIG. 2 was provided by critical CO₂ extraction.

Generally, crystalline CBD can be provided by methods and techniques known in the art, such as by methods disclosed in U.S. Ser. No. 10/413,845 and/or U.S. Ser. No. 10/414,709.

In short, crystalline CBD can be provided from hemp or cannabis (Cannabis sativa) by a method consisting essentially of:
Extracting hemp or cannabis with e.g. isopropanol to produce an extract rich in cannabinoids, THC, CBD and terpenes
Evaporating the solvent portion of the extract to generate a substantially solvent-free extract
Distilling the substantially solvent-free extract to isolate the CBD, and
Crystallizing the distilled, isolated CBD to produce a crystallized, isolated CBD and one or more recrystallization(s) if needed by the use of a suitable organic solvent, such as an alkane, e.g. heptane, commonly followed by
Solvent removal by e.g. vacuum drying. to remove volatile remnants.

Thus, in some embodiments, the CBD crystals used in the formulation of the topical composition are needle-like crystals, such as crystals shown in FIG. 1. Likewise, in some embodiments, the CBD crystals used in the formulation of the topical composition are not cluster- or bunch-shaped, such as crystals similar to crystals shown in FIG. 2.

In some embodiments, the CBD crystals used in the formulation of the topical composition are not provided by an extraction method comprising critical CO₂ extraction.

In some embodiments, the CBD crystals used in the formulation of the topical composition are provided by a method comprising extraction with a C₃-C₄ alcohol, such as isopropanol, and one or more crystallisations steps with a C₆-C₈ alkane, such as heptane. In some embodiments, the C₃-C₄ alcohol is isopropanol. In some embodiments, the C₆-C₈ alkane is heptane. In some embodiments, the C₃-C₄ alcohol is isopropanol, and the C₆-C₈ alkane is heptane. This combination is believed to provide CBD crystals of satisfactory quality, such as absence or reduction in inhibitors and/or the desired conformation of the CBD.

In some embodiments, a suitable CBD product can be obtained when the CBD crystals are provided by a method comprising critical CO₂ extraction and one or more crystallisations steps with a C₆-C₈ alkane, such as heptane.

As seen in Table 1, it can be seen that the Cannabinoid profile of type A and type B CBD can be rather similar.

TABLE 1
Analysis of CBD of crystal structure A versus crystal structure B

| Cannabinoid profile | Type A | Type B |
|---|---|---|
| CBD | 99.33% | 98.60% |
| CBDV | 0.39% | 0.19% |
| CBDA | 0.01% | n.d. |
| CBG | n.d. | n.d. |
| CBN | 0.04% | n.d. |
| THC | n.d. | n.d. |

n.d. not detected;
type A CBD was sourced from Enecta,
type B CBD was sourced from Pharma Hemp

It is, however, also conceivable that the differences in crystal structure, can relate to and be caused by different extraction processes. Different crystal structures can also be indicative of different concentrations of “CBD inhibitors”, and/or different concentrations of “CBD enhancers”. In some embodiments, terpenes, such as naturally occurring terpenes, in particular terpenes found in plants, such as in Cannabis sativa, act as CBD inhibitors, which is not desirable.

Thus, in some embodiments, CBD of crystal structure B alias “type B CBD” can be converted to CBD of crystal structure A alias “type A CBD” (and/or CBD capable of forming crystal structure A) by an organic extraction step and/or recrystallisation step. In such embodiments, it is conceivable that the change in crystal structure is related to the presence of inhibitors that are reduced significantly in the additional extraction and/or crystallization step(s). Alternatively, the organic extraction step may provide a change in conformation of the CBD, rendering it more active again. In some embodiments, recrystallization with heptane can change the B-type CBD into A-type CBD.

In some embodiments, CBD of crystal structure B has been provided by critical CO₂ extraction, such as CBD crystals provided by www.pharma-hemp.com and/or following a similar extraction protocol as said manufacturer.

In some embodiments, presence of terpenes and/or terpenoids, in particular Cannabis sativa terpenes or in a CBD-comprising topical composition as disclosed herein, provides one or more undesirable effect(s), such as one or more of: reduced efficiency or potency, inability or reduced ability to recognize the CBD, need for a higher CBD formulation for obtaining similar effect, increase in non-CBD cannabinoids in the formulation. In some embodiments, said composition comprises 0.0001% or less, 0.001% or less, 0.01% or less, or 0.1% or less terpenes, in particular Cannabis sativa terpenes, by weight.

In some embodiments, the crystalline CBD does not comprise significant amounts of terpenes, such as less than 0.1, less than 0.05, less than 0.02, less than 0.01, less than 0.005, less than 0.002, less than 0.001% terpenes by weight.

It is also conceivable that other plant components, such as terpenoids can act as inhibitors. In some embodiments the presence of terpenoids, such as Cannabis sativa terpenoids can be undesirable. In some embodiments, the crystalline CBD does not comprise significant amounts of terpenoids, such as less than 0.1, less than 0.05, less than 0.02, less than 0.01, less than 0.005, less than 0.002, less than 0.001% terpenoids by weight.

In some embodiments, the use of CBD having or capable of providing crystals of crystal structure A, such as shown in FIG. 1 in a CBD-comprising topical composition as disclosed herein, provides a positive effect, such as one or more of: increased efficiency, possibility to reduce total amount of CBD in the formulation, the subject needs less topical formulation to achieve the same effect, improved recognition and/or CBD uptake by the subject's body, reduction in non-CBD cannabinoids in the formulation and/or other impurities.

As already disclosed in the third aspect the present invention may also concern methods of treatment. In particular, in a fourth aspect, the present invention concerns a method of treatment and/or alleviation of symptoms such as pain and/or discomfort in a subject, such as pain or discomfort related to a disease and/or condition, comprising e.g. autoimmune-related diseases, stress related conditions, such as one or more of: arthritis, rheumatoid arthritis, osteoarthritis, juvenile rheumatoid arthritis, psoriatic arthritis and/or neurological pain, such as pain resulting from sclerosis, psoriasis, multiple sclerosis (MS), herpes, such as herpes zoster, seborrheic dermatitis, eczema, neurodermitis, Lichen simplex chronicus (LSC), atopic dermatitis (AD) aka atopic eczema, Lupus, such as systemic lupus erythematosus (SLE), scleroderma, urticaria, a painful condition associated with one or more hot and/or swollen joints, and/or a skin-, nerve-, joint condition; wherein said method comprises the use of a supplement according to any one of the preceding aspects.

In some embodiments, said treatment comprises the use of a topical composition comprising a cannabinoid, such as cannabinol (CBD). Said composition(s) can be e.g. be formulated as a hydroalcoholic gel, such as an arthritis CBD gel, or a psoriasis CBD gel, e.g. according to the third aspect or the Examples presented herein.

In some embodiments, a method of treatment and/or alleviation of symptoms is disclosed, concerning e.g. pain and/or discomfort in a subject, such as a subject as defined herein, wherein said pain and/or discomfort relates to a disease and/or condition, comprising or related to autoimmune-related diseases, stress related conditions, such as one or more of: arthritis, rheumatoid arthritis, osteoarthritis, juvenile rheumatoid arthritis, psoriatic arthritis and/or neurological pain, such as pain resulting from sclerosis, psoriasis (e.g. in particular red, dry, itchy, and/or scaly skin), multiple sclerosis (MS), herpes, such as herpes zoster, seborrheic dermatitis, eczema, neurodermitis, Lichen simplex chronicus (LSC), atopic dermatitis (AD) aka atopic eczema, Lupus, such as systemic lupus erythematosus (SLE), scleroderma, urticaria, a painful condition associated with one or more hot and/or swollen joints, and/or a skin-, nerve-, joint condition; wherein said method comprises the use of an oral composition, such a supplement according to any one of the preceding aspects.

In some embodiments, the treatment and/or alleviation of symptoms are related to arthritis or psoriasis, and/or arthritis- and/or psoriasis-related conditions or symptoms.

In some embodiments, said supplement/composition is a composition according to the first aspect, and said treatment is related to arthritis. In some embodiments, the composition is an “arthritis composition” as disclosed herein, e.g. in any one of claims 1-3, 5-10, 13, 16-18. Generally, an “arthritis composition” will provide one or more beneficial effects in comparison to a “psoriasis composition”, when used in the treatment of arthritis-related conditions.

In some embodiments, said supplement/composition is a composition according to the first aspect, and said treatment is related to psoriasis. In some embodiments, the composition is a “psoriasis composition” as disclosed herein, such as in any one of claims 1-2, 4-8, 11-12, 14-18. Generally, a “psoriasis composition” will provide one or more beneficial effects in comparison to an “arthritis composition”, when used in the treatment of arthritis-related conditions.

In some embodiments, the treatment may further comprise the use of a topical composition, such as a topical composition for treatment of arthritis or psoriasis. Generally, it is believed that the effect of combined treatment (oral composition+topical composition provides one or more beneficial effects compared to a treatment with oral composition alone.

In some embodiments, said topical composition is a cannabinoid-comprising topical composition. In some embodiments, the topical composition is a composition as disclosed e.g. in the third aspect of the invention. In some embodiments, the topical composition may comprise one or more cannabinoids, such as CBD. Thus in some embodiments, the method of treatment comprises the use of an oral composition in combination with the use of a cannabinoid-comprising topical composition.

In some embodiments, the method of treatment comprises the use of a CBD-comprising hydroalcoholic gel, such as a hydroalcoholic gel as disclosed herein, such as in the third aspect. In some embodiments, said treatment comprises the use of a CBD-comprising gel as disclosed herein, and/or in WO2021023351.

In some embodiments, the method of treatment comprises the use of an “arthritis composition/supplement” as disclosed herein, and a CBD-comprising hydroalcoholic gel formulated for treatment of arthritis.

In some embodiments, the method of treatment comprises the use of an “psoriasis composition/supplement” as disclosed herein, and a CBD-comprising hydroalcoholic gel formulated for treatment of arthritis, such as disclosed herein and/or in WO2021023351.

In a fifth aspect, the present invention relates to a receptacle comprising a composition according to any one of the preceding aspects.

In some embodiments, a receptacle is provided comprising a composition, such as an oral composition. In some embodiments, the receptacle comprises a supplement, such as an “arthritis supplement” or “psoriasis supplement”.

In some embodiments, the receptacle provides protection from visible and/or UV-light. Suitable materials for providing receptacles, and in particular receptacles providing light-protection are known in the art.

In some embodiments, a receptacle is provided comprising a topical composition as disclosed herein, e.g. according to the third or fourth aspect, or in the Examples. In some embodiments, said receptacle comprises topical composition for treatment of a condition, disorder, or symptom as disclosed herein, such as a cannabinoid-comprising topical composition, e.g. a CBD alcoholic gel, such as an “arthritis CBD gel” or a “psoriasis CBD gel”, in particular, but not exclusively, an “arthritis hydroalcoholic CBD gel” or a “psoriasis hydroalcoholic CBD gel”.

In a sixth aspect, the present invention pertains to a kit comprising a receptacle according to the fifth aspect, an instruction for use, and optionally a packaging.

In some embodiments, said kit may comprise more than one receptacle, such as at least one receptacle comprising an oral composition, and at least one receptacle comprising a topical composition.

In a seventh aspect, the present invention concerns a CBD-comprising composition, such as a topical composition, wherein the CBD used in the formulation is crystalline. In some embodiments, said composition is a topical composition as disclosed herein, and/or in the third and/or fourth aspect. In some embodiments, the CBD is of type A (needle-like crystals) or capable of forming needle-like crystals. as disclosed in the third and/or fourth aspect.

In an eiaht aspect, the present invention pertains to a dosage regimen, comprising administering an oral supplement as disclosed herein in combination with a topical composition, in particular CBD-comprising topical composition as disclosed herein.

In summary, daily intake of compositions and/or supplements as disclosed herein may provide significant benefits.

Daily intake of an arthritis composition/supplement as disclosed herein may provide one or more of the following benefits, advantages or effects, including any combination(s) thereof: anti-inflammatory, anti-arthritic, and/or analgesic effect; relieve pain and/or stiffness; improves function of one or more joints; improved mobility; fewer side effects; reduction in overweight; anti-cancer; neuroprotective; reduces progression of osteoarthritis; reduction in osteoarthritis-associated pain and/or discomfort; anti-nociceptive and/or anti-arthritic effect; repairment of own joint cartilage; protects against joint damage; provides symptom relief; support for tired, overworked joints; support joint comfort; improved/increased range of motion; improved body mobility; composition triggers the body to rebuild and/or repair joint cartilage.; Helps the body's natural repair mechanisms in the joints; Prolonged maintenance of healthy joint during aging; Maintained mobility with age; lengthen the period of pain free strenuous exertion and alleviate the joint pain that occasionally arises from such activities.; improve joint extension/flexion and/or mobility; collagen is rebuild in a normal fashion; normal function of bones and joints; reduction in bone-degradation-improved bone health; and/or action against auto-immune conditions.

Daily intake of a psoriasis composition/supplement may provide one or more of the following effects, including any combination(s) thereof: reduction of psoriasis-related symptoms of one or more of: red, dry, itchy, and/or scaly skin; anti-inflammatory and analgesic agent; Anti psoriasis; Anti seborrheic dermatitis; Reduces too rapidly growing skin cells; Reduce redness and/or swelling; Skin healing; Positive action(s) on skin biology and/or psoriasis pathogenesis; Regulations of keratinocytes proliferation, differentiation and/or apoptosis; Regulation of cutaneous immune system (inhibition of T cell proliferation, Tregs induction); Down-regulation of pro-inflammatory cytokines; Stimulation of antimicrobial peptides expression; Regulation of barrier integrity and permeability; Anti-stress; Anti-Psoriatic Arthritis; Anti-oxidative stress; Improved wound healing; Improved UV-protection; Improved microbial flora, e.g. gut and/or skin flora; Anti-skin cancer; Anti-autoimmune illness; and/or inhibition of MAPK-dependent signalling pathways, and stimulation of epidermal proliferation, differentiation, and lipid production.

A daily intake of a composition as disclosed herein, such as a composition/supplement according to the first aspect of the invention, in particular an “arthritis composition” combined with application of topical composition for treatment of arthritis, such as a CBD-comprising composition, in particular a CBD-comprising hydroalcoholic gel, such as an arthritis CBD gel may provide one or more further effects, including any combination(s) thereof: Cooling therapy; Anti-autoimmune illness; Pain relief; Better mobility; More active lifestyle; Down-regulation of pro-inflammatory cytokines; Reduction of need for NSAID medicine and/or reduction in side effects caused by NSAID medicine; and/or improved liver count.

A daily intake of supplement as disclosed herein, such as a supplement according to the first aspect of the invention, in particular a “psoriasis composition” combined with application of topical composition for treatment of psoriasis, such as a CBD-comprising composition, in particular a CBD-comprising hydroalcoholic gel, such as a psoriasis CBD gel provides one or more further effects, including any combination(s) thereof: Immediately itch relief—faster healing; recovery/healing from psoriasis; action against autoimmune illness; pain relief; and/or down-regulation of pro-inflammatory cytokines.

Generally, a combination treatment (oral+topical composition) may provide an increased or stronger effect of any one of the effects disclosed of an oral composition or supplement alone, such as those disclosed above.

Furthermore, it is believed that subject may achieve a symptom-free, or near symptom free condition through combination treatment with a supplement and a cannabinol-comprising topical composition, thereby increasing their quality of life of a subject/patient.

Hereinafter, the present invention is described in more detail and specifically with reference to the Examples, which however are not intended to limit the present invention.

### EXAMPLES

### Example 1—Provision of Supplements

Supplements according to the present invention are provided using methods and know-how customary in the field.

All supplements are formulated as ˜720 mg capsules using Rice Bran Extract as excipient (up to 100% by weight), and a capsule shell (˜17% by weight) comprising hydroxypropyl methyl cellulose as glazing agent and TiO₂ as colouring agent). Maltodextrin is used in the placebo formulation.

Supplements 1-3 are formulated as a single dose/capsule per day; 1 capsule provides the daily required dose of ingredients.

Supplement 4 is formulated requiring 2 capsules per day, such that 2 capsules provide the daily required dos of ingredients.

Supplement 5 is formulated requiring 3 capsules per day, such that 3 capsules provide the daily required dos of ingredients.

Supplement 1
4% by weight 3-O-Acetyl-11-Keto Beta Boswellic Acid (AKBBA);
8% by weight Curcuminoid(s);
6% by weight L-Ascorbic acid (Vitamin C);
0.00035% by weight Cholecalciferol (Vitamin D); and/or
0.7% by weight Piperidine.

Supplement 1 is formulated as follows:

| | mg active | % active | mg per |
|---|---|---|---|
| Ingredients Supplement 1 | ingredient | ingredient | capsule |
| Boswellia Serrata Resin Extract | 30 | 30 | 100 |
| (Boswellia serrata) [30% AKBBA] | | | |
| Curcuma Longa Rhizomes Extract | 60 | 90 | 66.67 |
| (Curcuma longa)[90% Curcuminoids] | | | |
| Rosehip Fruit Extract (Rosa canina) | 45 | 70 | 64.29 |
| [70% Vitamin C] | | | |
| Black Pepper Fruit Extract (Piper | 4.75 | 95 | 5 |
| nigrum) [95% Piperine] | | | |
| Cholecalciferol (Vitamin D - Vita-algae | 0.0025 | 0.185 | 1.35 |
| D ®)(0.185% vit. D) | | | |
| Rice Bran Extract | | 100 | 360.69 |
| Capsule Shell | | 100 | 122 |
| Glazing Agent: Hydroxypropyl | (119.56) | | |
| Methylcellulose | | | |
| Colour: Titanium Dioxide | (2.44) | | |
| (E-171) | | | |
| | | total | 720 |

Supplement 2
4% by weight 3-O-Acetyl-11-Keto Beta Boswellic Acid (AKBBA);
8% by weight Curcuminoid(s);
6% by weight L-Ascorbic acid (Vitamin C);
0.00035% by weight Cholecalciferol (Vitamin D);
0.7% by weight Piperidine;
9% by weight Bromelain (1200 GDU/g); and
1.4% by weight Collagen

formulated as 720 mg capsule:

| | mg active | % active | mg per |
|---|---|---|---|
| Ingredients Supplement 2 | ingredient | ingredient | capsule |
| Boswellia Serrata Resin Extract | 30 | 30 | 100 |
| (Boswellia serrata) [30% AKBBA] | | | |
| Bromelain from Pineapple (Ananas | 65 | 65 | 100 |
| comosus) (1200 GDU/g) | | | |
| Curcuma Longa Rhizomes Extract | 60 | 90 | 66.67 |
| (Curcuma longa)[90% Curcuminoids] | | | |
| Rosehip Fruit Extract (Rosa canina) | 45 | 70 | 64.29 |
| [70% Vitamin C] | | | |
| UC-II ® - Standardized Chicken | 10 | 25 | 40 |
| Cartilage Powder [25% Total | | | |
| Collagen] | | | |
| Black Pepper Fruit Extract (Piper | 4.75 | 95 | 5 |
| nigrum) [95% Piperine] | | | |
| Cholecalciferol (Vitamin D - Vita- | 0.0025 | 0.185 | 1.35 |
| algae D ®)(0.185% vit. D) | | | |
| Rice Bran Extract | | 100 | 220.7 |
| Capsule Shell | | 100 | 122 |
| Glazing Agent: Hydroxypropyl | (119.56) | | |
| Methylcellulose | | | |
| Colour: Titanium Dioxide (E-171) | (2.44) | | |
| | | total | 720.01 |

Supplement 3
2% by weight 3-O-Acetyl-11-Keto Beta Boswellic Acid (AKBBA);
4% by weight Curcuminoid(s);
3% by weight L-Ascorbic acid (Vitamin C);
0.01% by weight Cholecalciferol (Vitamin D); and
2.8% by weight Hesperidin.

Formulated as 720 mg capsule:

| | mg active | % active | mg per |
|---|---|---|---|
| Ingredients | ingredient | ingredient | capsule |
| Boswellia Serrata Resin Extract | 30 | 30 | 100 |
| (Boswellia serrata) [30% AKBBA] | | | |
| Curcuma Longa Rhizomes Extract | 58 | 90 | 52.2 |
| (Curcuma longa)[90% | | | |
| Curcuminoids] | | | |
| Rosehip Fruit Extract (Rosa | 44 | 70 | 30.8 |
| canina) [70% Vitamin C] | | | |
| Bitter Orange Peel Extract (Citrus | 41 | 90 | 36.9 |
| aurantium) [90% Hesperidin] | | | |
| Cholecalciferol (Vitamin D - Vita- | 0.15 | 0.185 | 0.000278 |
| algae D ®)(0.185% vit. D) | | | |
| Rice Bran Extract | 378.1 | 100 | 378.1 |
| Capsule Shell | 122 | 100 | 122 |
| Glazing Agent: Hydroxypropyl | (119.56) | | |
| Methylcellulose | | | |
| Colour: Titanium Dioxide (E-171) | (2.44) | | |
| | | total | 720.0003 |

Supplement 4 (2 capsules per day)
2% by weight 3-O-Acetyl-11-Keto Beta Boswellic Acid (AKBBA);
4% by weight Curcuminoid(s);
3% by weight L-Ascorbic acid (Vitamin C);
0.01% by weight Cholecalciferol (Vitamin D);
2.8% by weight Hesperidin;
0.5% by weight Rosavin;
1.0% by weight Zinc gluconate;
0.07% by weight Vitamin B12;
0.5% by weight Withanolides;
0.07% by weight Vitamin A;
0.003% by weight Selene; and
1.25% by weight Probiotic bacteria (5×10¹⁰ CFU/g)

Formulated as 720 mg capsule:

| | mg active | % active | mg per |
|---|---|---|---|
| Ingredients | ingredient | ingredient | capsule |
| Boswellia Serrata Resin Extract | 15 | 30 | 50 |
| (Boswellia serrata) [30% AKBBA] | | | |
| Curcuma Longa Rhizomes Extract | 30 | 90 | 33.33 |
| (Curcuma longa) [90% | | | |
| Curcuminoids] | | | |
| L-Ascorbic Acid (Vitamin C) | 22.5 | 100 | 22.5 |
| Cholecalciferol (Vitamin D) | 0.1 | 0.185 | 54 |
| Bitter Orange Peel Extract (Citrus | 20 | 90 | 22.22 |
| aurantium) [90% Hesperidin] | | | |
| Rhodiola Rosea Root Extract | 2.4 | 3 | 125 |
| (Rhodiola rosea) [3% Rosavin] | | | |
| Zinc Gluconate - Zinc | 7.5 | 9.23 | 81.25 |
| Cyanocobalamin (Vitamin B12) | 0.5 | 1 | 50 |
| Ashwagandha Root Extract | 3.5 | 7 | 50 |
| (Withania somnifera) [7% | | | |
| Withanolides] | | | |
| Retinyl Acetate (Vitamin A) | 0.5 | 7.8 | 6.41 |
| L-Selenomethionine- | 0.02 | 0.39 | 5.2 |
| SeleniumSeLECT ® | | | |
| Methylocobalamin (Vitamin B12) | 0.45 | 100 | 0.45 |
| Probiotic Bacillus coagulans | 9 | 90 | 10 |
| ATCC7050 (50 Billion CFU/g) | | | |
| Rice Bran Extract | 87.63 | 100 | 87.63 |
| Capsule Shell | 122 | 100 | 122 |
| Glazing Agent: Hydroxypropyl | (119.56) | | |
| Methylcellulose | | | |
| Colour: Titanium Dioxide (E-171) | (2.44) | | |
| | | Total | 719.99 |

Supplement 5 (3 capsules/day)
1.4% by weight 3-O-Acetyl-11-Keto Beta Boswellic Acid (AKBBA); or
2.8% by weight Curcuminoid(s);
2% by weight L-Ascorbic acid (Vitamin C);
0.01% by weight Cholecalciferol (Vitamin D);
1.85% by weight Hesperidin;
0.35% by weight Rosavin;
0.7% by weight Zinc gluconate;
0.05% by weight Vitamin B12;
0.3% by weight Withanolides;
0.05% by weight Vitamin A;
0.002% by weight Selene;
8.3% by weight L-cysteine HCL Mono;
9.0% by weigh L-glutamine;
20% omega 3 fatty acids
1.25% by weight Probiotic bacteria (50×10⁹ CFU/g).

Formulated as 720 mg capsule:

| | mg active | % active | mg per |
|---|---|---|---|
| Ingredients | ingredient | ingredient | capsule |
| Rhodiola Rosea Root Extract | 2.5 | 3 | 83.33 |
| (Rhodiola rosea) [3% Rosavin] | | | |
| Zinc Gluconate - Zinc | 5 | 9.23 | 54.17 |
| Cholecalciferol (Vitamin D) | 0.07 | 0.185 | 36.00 |
| Cyanocobalamin (Vitamin B12) | 0.33 | 1 | 33.33 |
| Ashwagandha Root Extract | 2.33 | 7 | 33.33 |
| (Withania somnifera) [7% | | | |
| Withanolides] | | | |
| Boswellia Serrata Resin Extract | 10 | 30 | 33.33 |
| (Boswellia serrata) [30% AKBBA] | | | |
| Curcuma Longa Rhizomes Extract | 20 | 90 | 22.22 |
| (Curcuma longa) [90% | | | |
| Curcuminoids] | | | |
| L-Ascorbic Acid (Vitamin C) | 15 | 100 | 15.00 |
| Bitter Orange Peel Extract (Citrus | 13.33 | 90 | 14.81 |
| aurantium) [90% Hesperidin] | | | |
| Probiotic Bacillus coagulans | 6 | 90 | 6.67 |
| ATCC7050 (50 Billion CFU/g) | | | |
| Retinyl Acetate (Vitamin A) | 0.33 | 7.8 | 4.27 |
| L-Selenomethionine- | 0.02 | 0.39 | 3.47 |
| SeleniumSeLECT ® | | | |
| Methylocobalamin (Vitamin B12) | 0.3 | 100 | 0.30 |
| L-cysteine HCL Mono | 60 | 90 | 66.67 |
| L-glutamine | 65.3 | 98 | 66.67 |
| Omega 3 | 13.3 | 20 | 66.67 |
| Capsule Shell | 122.00 | 100 | 122.00 |
| Glazing Agent: Hydroxypropyl | (119.56) | | |
| Methylcellulose | | | |
| Colour: Titanium Dioxide (E-171) | (2.44) | | |
| Rice Bran Extract | 57.75 | 100 | 57.75 |
| | | total | 719.99 |

Supplement 6—placebo (720 mg capsule)
Maltodextrin DE 15-20: 510 mg
Rice bran extract: 88 mg
Capsule shell: 122 mg

### Example 2—Provision of CBD-Comprising Compositions

CBD-comprising compositions according to the present invention can be provided using methods and/or know-how customary in the field. Concerning CBD-hydroalcoholic gels, these are disclosed in DK priority application PA 2019 70497, DK priority application PA 2020 70343, and/or WO2021023351.

CBD-Hydroalcoholic Gel 1 (“Arthritis Gel”)

Arthritis CBD-hydroalcoholic gels can be provided essentially as disclosed in WO2021023351, Example 2, using type A crystalline CBD from Enecta.

CBD-Hydroalcoholic Gel 2 (“Psoriasis Gel”)

Psoriasis CBD-hydroalcoholic gels can be provided essentially as disclosed in WO2021023351, Example 1, using type A crystalline CBD from Enecta.

### Example 3—Intake of Supplement

Generally, supplements are consumed in the morning, preferably in combination with breakfast, and consuming around 100 ml fluid or more.

### Example 4—Application of Topical Composition

Generally, topical applications are applied twice a day (morning and evening). CBD-alcoholic gels are applied according to the instructions for use, such as disclosed in Example 11 and 12 of WO2021023351.

### Example 5—Combination Treatment (Supplement+Topical Composition)

Combination treatments comprise intake of supplements in the morning (see Example 3, and application of CBD-alcoholic gels in the morning and evening.

### Example 6—Test Set-Up

### I. Supplements

Arthritis test & Placebo groups-Inclusion criteria:
People with arthritis
only using NSAID (nonsteroidal anti-inflammatory drug) for pain relief
8 persons per group
age 30-80.

Psoriasis test & Placebo groups-Inclusion criteria:
People with psoriasis
only using NSAID for pain relief
6 persons per group
age 20-80.

### II. Topical Compositions

Arthritis test & Placebo groups—Inclusion criteria:
People with arthritis
only using NSAID for pain relief
8 persons per group
age 30-80.

Psoriasis test & Placebo groups—Inclusion criteria:
People with Psoriasis
only using NSAID for pain relief
6 persons per group
age 20-80.

### III. Supplement+Topical Composition

Arthritis test & Placebo groups—Inclusion criteria:
People with arthritis
only using NSAID for pain relief
8 persons per group
age 30-80.

Psoriasis test & Placebo groups-Inclusion criteria:
People with Psoriasis
only using NSAID for pain relief
6 persons per group
age 20-80.

Results are presented in the following Examples.

### Example 7—Results Arthritis Supplements

TABLE A
Supplement 1

| Testperson | 1 | 2 | 3 | 4 | 5 | 6 | 7 | 8 |
|---|---|---|---|---|---|---|---|---|
| Typical range of | 0-140 | 0-145 | 0-140 | 0-90 | 75-80 | 0-50 | 0-145 | 0-140 |
| motion | degrees | degrees | degrees | degrees | degrees | degrees | degrees | degrees |
| Joint with symptoms | Knee | Elbow | Knees | Fingers | Wrists | Ankle | Elbow | Knees |
| of arthritis | | | | | | | | |
| Test period(days) | 110 | 97 | 170 | 140 | 120 | 130 | 150 | 180 |
| minimum 90 days-max | | | | | | | | |
| 180 days | | | | | | | | |
| Joint | B: 110 | B: 115 | B: 120 | B: 70 | B: 60 | B: 35 | B: 115 | B: 100 |
| extension/flexion | A: 90 | A: 98 | A: 70 | A: 40 | A: 50 | A: 20 | A: 90 | A: 45 |
| in degrees ° | | | | | | | | |
| before and after | | | | | | | | |
| test period | | | | | | | | |
| Pain scaled on a | B: 8 | B: 7 | B: 8 | B: 9 | B: 6 | B: 7 | B: 7 | B: 8 |
| 0-10 scale before | A: 6 | A: 5 | A: 5 | A: 6 | A: 4 | A: 5 | A: 4 | A: 4 |
| and after test period | | | | | | | | |
| Pain free exercise | B: 0 | B: 1 | B: 0 | B: 0 | B: 1 | B: 1 | B: 1 | B: 0 |
| in min. before and | A: 12 | A: 15 | A: 18 | A: 15 | A: 12 | A: 14 | A: 17 | A: 20 |
| after test period | | | | | | | | |
| Medication reduced | NA | NA | NA | NA | NA | NA | NA | NA |
| during test period? | | | | | | | | |

TABLE B
Supplement 2

| Testperson | 1 | 2 | 3 | 4 | 5 | 6 | 7 | 8 |
|---|---|---|---|---|---|---|---|---|
| Typical range of | 0-140 | 0-145 | 0-90 | 75-80 | 0-50 | 0-140 | 0-145 | 75-80 |
| motion | degrees | degrees | degrees | degrees | degrees | degrees | degrees | degrees |
| Joint with symptoms | Right | Left | Fingers | Left | Ankle | Left | Right | Left |
| of arthritis | Knee | Elbow | | Wrist | | Knee | Elbow | Wrist |
| Test period(days) | 180 | 160 | 140 | 120 | 110 | 100 | 90 | 95 |
| minimum 90 days-max | | | | | | | | |
| 180 days | | | | | | | | |
| Joint | B: 100 | B: 110 | B: 70 | B: 60 | B: 35 | B: 125 | B: 130 | B: 68 |
| extension/flexion | A: 20 | A: 40 | A: 40 | A: 38 | A: 20 | A: 102 | A: 115 | A: 50 |
| in degrees ° | | | | | | | | |
| before and after | | | | | | | | |
| test period | | | | | | | | |
| Pain scaled on a | B: 10 | B: 9 | B: 8 | B: 7 | B: 8 | B: 9 | B: 8 | B: 9 |
| 0-10 scale before | A: 4 | A: 5 | A: 4 | A: 4 | A: 5 | A: 6 | A: 7 | A: 7 |
| and after test period | | | | | | | | |
| Pain free exercise | B: 0 | B: 0 | B: 1 | B: 0 | B: 1 | B: 1 | B: 0 | B: 0 |
| in min. before and | A: 30 | A: 25 | A: 20 | A: 15 | A: 14 | A: 12 | A: 11 | A: 10 |
| after test period | | | | | | | | |
| Medication reduced | NA | Yes | Yes | NA | NA | Yes | Yes | Yes |
| during test period? | | | | | | | | |

TABLE C
Supplement 6 (Placebo)

| Testperson | 1 | 2 | 3 | 4 | 5 | 6 | 7 | 8 |
|---|---|---|---|---|---|---|---|---|
| Joint with symptoms | Elbow | Knee | Fingers | Wrists | Ankle | Knees | Elbow | Wrists |
| of arthritis | | | | | | | | |
| Typical range of | 0-145 | 0-140 | 0-90 | 75-80 | 0-50 | 0-140 | 0-145 | 75-80 |
| motion | degrees | degrees | degrees | degrees | degrees | degrees | degrees | degrees |
| Test period(days) | 95 | 102 | 110 | 118 | 125 | 135 | 165 | 180 |
| minimum 90 days-max | | | | | | | | |
| 180 days | | | | | | | | |
| Joint | B: 135 | B: 120 | B: 88 | B: 66 | B: 48 | B: 120 | B: 123 | B: 55 |
| extension/flexion | A: 134 | A: 120 | A: 86 | A: 65 | A: 46 | A: 120 | A: 122 | A: 55 |
| in degrees ° | | | | | | | | |
| before and after | | | | | | | | |
| test period | | | | | | | | |
| Pain scaled on a | B: 8 | B: 7 | B: 9 | B: 7 | B: 8 | B: 9 | B: 8 | B: 8 |
| 0-10 scale before | A: 8 | A: 7 | A: 8 | A: 5 | A: 7 | A: 9 | A: 8 | A: 7 |
| and after test period | | | | | | | | |
| Pain free exercise | B: 0 | B: 0 | B: 0 | B: 0 | B: 0 | B: 0 | B: 0 | B: 0 |
| in min. before and | A: 0 | A: 0 | A: 2 | A: 1 | A: 1 | A: 0 | A: 1 | A: 1 |
| after test period | | | | | | | | |
| Medication reduced | NA | NA | NA | NA | NA | No | No | NA |
| during test period? | | | | | | | | |

### Example 8—Psoriasis Supplement

TABLE D
Supplement 3 (1 capsules a day)

| Testperson | 1 | 2 | 3 | 4 | 5 | 6 |
|---|---|---|---|---|---|---|
| Psoriasis spot | Hand - | Hairline | Elbow - | Hand - | Scalp | Feet - |
| size and position | 3*3 cm | neck - | 5*5 cm | 1*2 cm | 1*2 cm | 4*2 cm |
| | | 2*8 cm | | | | |
| Test period(days) | 110 | 97 | 170 | 180 | 90 | 180 |
| minimum 90 days-max | | | | | | |
| 180 days | | | | | | |
| Ich scaled on a 0-10 | B: 7 | B: 8 | B: 9 | B: 7 | B: 9 | B: 8 |
| scale before and | A: 5 | A: 7 | A: 6 | A: 4 | A: 8 | A: 4 |
| after test period | | | | | | |
| Redness & Swelling | B: 8 | B: 10 | B: 9 | B: 7 | B: 9 | B: 7 |
| scaled on a 0-10 | A: 5 | A: 8 | A: 6 | A: 4 | A: 8 | A: 4 |
| scale before and | | | | | | |
| after test period | | | | | | |
| Psoriasis spot | −3*2 cm | −2*8 cm | −5*3 cm | −1*1 cm | 1*2 cm | −3*2 cm |
| size in cm after | | | | | | |
| test period | | | | | | |
| Medication reduced | None | None | None | None | None | None |
| during test period? | taking | taking | taking | taking | taking | taking |

TABLE E
Supplement 4 (2 capsules/day)

| Testperson | 1 | 2 | 3 | 4 | 5 | 6 |
|---|---|---|---|---|---|---|
| Psoriasis spot | Stomach - | Armpits - | Back - | Leg - | Knee - | Hand - |
| size and position | 3*4 cm | 2*4 cm | 6*5 cm | 2*5 cm | 4*4 cm | 1*5 cm |
| Test period(days) | 100 | 90 | 180 | 140 | 120 | 100 |
| minimum 90 days-max | | | | | | |
| 180 days | | | | | | |
| Ich scaled on a 0-10 | B: 9 | B: 10 | B: 9 | B: 8 | B: 8 | B: 8 |
| scale before and | A: 4 | A: 5 | A: 4 | A: 4 | A: 6 | A: 4 |
| after test period | | | | | | |
| Redness & Swelling | B: 8 | B: 10 | B: 10 | B: 9 | B: 6 | B: 7 |
| scaled on a 0-10 | A: 5 | A: 6 | A: 5 | A: 4 | A: 4 | A: 3 |
| scale before and | | | | | | |
| after test period | | | | | | |
| Psoriasis spot | −2*4 cm | −2*3 cm | −5*3 cm | −1*5 cm | 3*4 cm | 1*2 cm |
| size in cm after | | | | | | |
| test period | | | | | | |
| Medication reduced | None | None | None | None | None | None |
| during test period? | taking | taking | taking | taking | taking | taking |

TABLE F
Supplement 5 (3 capsules/day)

| Testperson | 1 | 2 | 3 | 4 | 5 | 6 |
|---|---|---|---|---|---|---|
| Psoriasis spot | Hairline | Scalp | Stomach - | Elbow - | Feet - | Armpits - |
| size and position | neck - | 1*4 cm | 5*5 cm | 3*5 cm | 4*2 cm | 2*4 cm |
| | 2*10 cm | | | | | |
| Test period(days) | 180 | 100 | 170 | 140 | 90 | 120 |
| minimum 90 days-max | | | | | | |
| 180 days | | | | | | |
| Ich scaled on a 0-10 | B: 9 | B: 8 | B: 10 | B: 7 | B: 9 | B: 10 |
| scale before and | A: 2 | A: 4 | A: 2 | A: 2 | A: 4 | A: 4 |
| after test period | | | | | | |
| Redness & Swelling | B: 8 | B: 8 | B: 9 | B: 6 | B: 8 | B: 10 |
| scaled on a 0-10 | A: 2 | A: 4 | A: 2 | A: 2 | A: 3 | A: 2 |
| scale before and | | | | | | |
| after test period | | | | | | |
| Psoriasis spot | −1*10 cm | −1*2 cm | −5*2 cm | 1*5 cm | 1*2 cm | 2*2 cm |
| size in cm after | | | | | | |
| test period | | | | | | |
| Medication reduced | None | None | None | None | None | None |
| during test period? | taking | taking | taking | taking | taking | taking |

TABEL H
Supplement 6 (Placebo)

| Testperson | 1 | 2 | 3 | 4 | 5 | 6 |
|---|---|---|---|---|---|---|
| Psoriasis spot | Hand - | Scalp | Elbow - | Knee - | Stomach - | Armpits - |
| size and position | 3*5 cm | 2*4 cm | 4*5 cm | 5*4 cm | 4*5 cm | 2*4 cm |
| Test period(days) | 90 | 180 | 120 | 95 | 100 | 140 |
| minimum 90 days-max | | | | | | |
| 180 days | | | | | | |
| Ich scaled on a 0-10 | B: 7 | B: 8 | B: 9 | B: 7 | B: 7 | B: 10 |
| scale before and | A: 7 | A: 7 | A: 9 | A: 7 | A: 7 | A: 10 |
| after test period | | | | | | |
| Redness & Swelling | B: 9 | B: 10 | B: 9 | B: 8 | B: 8 | B: 10 |
| scaled on a 0-10 | A: 9 | A: 10 | A: 9 | A: 8 | A: 7 | A: 10 |
| scale before and | | | | | | |
| after test period | | | | | | |
| Psoriasis spot | −3*5 cm | 2*4 cm | −4*5 cm | 5*4 cm | −4*5 cm | −2*4 cm |
| size in cm after | | | | | | |
| test period | | | | | | |
| Medication reduced | None | None | None | None | None | None |
| during test period? | taking | taking | taking | taking | taking | taking |

### Exmaple 9—Arthritis Combination Treatment

TABEL I
Supplement 1 & Arthritis Gel

| Testperson | 1 | 2 | 3 | 4 | 5 | 6 | 7 | 8 |
|---|---|---|---|---|---|---|---|---|
| Typical range of | 0-140 | 0-145 | 0-140 | 0-90 | 75-80 | 0-50 | 0-140 | 0-90 |
| motion | degrees | degrees | degrees | degrees | degrees | degrees | degrees | degrees |
| Joint with symptoms | Knee | Elbow | Knees | Fingers | Wrists | Ankle | Knee | Fingers |
| of arthritis | | | | | | | | |
| Test period(days) | 145 | 125 | 180 | 180 | 165 | 120 | 180 | 150 |
| minimum 90 days-max | | | | | | | | |
| 180 days | | | | | | | | |
| Joint | B: 110 | B: 90 | B: 100 | B: 60 | B: 75 | B: 45 | B: 130 | B: 85 |
| extension/flexion | A: 60 | A: 45 | A: 65 | A: 30 | A: 45 | A: 25 | A: 100 | A: 65 |
| in degrees ° | | | | | | | | |
| before and after | | | | | | | | |
| test period | | | | | | | | |
| Pain scaled on a | B: 8 | B: 7 | B: 9 | B: 7 | B: 9 | B: 5 | B: 8 | B: 6 |
| 0-10 scale before | A: 4 | A: 3 | A: 3 | A: 1 | A: 4 | A: 2 | A: 4 | A: 1 |
| and after test period | | | | | | | | |
| Pain free exercise | B: 0 | B: 0 | B: 5 | B: 0 | B: 10 | B: 0 | B: 0 | B: 5 |
| in min. before and | A: 30 | A: 25 | A: 35 | A: 20 | A: 45 | A: 20 | A: 35 | A: 20 |
| after test period | | | | | | | | |
| Medication reduced | Yes after | NA | NA | NA | Yes after | NA | Yes after | NA |
| during test period? | 2 weeks | | | | 5 weeks | | 8 weeks | |

TABEL J
Supplement 2 & Arthritis Gel

| Testperson | 1 | 2 | 3 | 4 | 5 | 6 | 7 | 8 |
|---|---|---|---|---|---|---|---|---|
| Typical range of | 0-140 | 0-145 | 75-80 | 0-140 | 75-80 | 0-90 | 0-145 | 0-50 |
| motion | degrees | degrees | degrees | degrees | degrees | degrees | degrees | degrees |
| Joint with symptoms | Left knee | Right | Right | Right | Left Wrist | Fingers | Left | Ankle |
| of arthritis | | Elbow | Wrist | Knee | | | Elbow | |
| Test period(days) | 96 | 180 | 155 | 170 | 100 | 120 | 143 | 105 |
| minimum 90 days-max | | | | | | | | |
| 180 days | | | | | | | | |
| Joint | B: 120 | B: 140 | B: 60 | B: 125 | B: 60 | B: 72 | B: 134 | B: 39 |
| extension/flexion | A: 26 | A: 10 | A: 17 | A: 13 | A: 25 | A: 21 | A: 18 | A: 23 |
| in degrees ° | | | | | | | | |
| before and after | | | | | | | | |
| test period | | | | | | | | |
| Pain scaled on a | B: 8 | B: 9 | B: 8 | B: 8 | B: 7 | B: 6 | B: 8 | B: 9 |
| 0-10 scale before | A: 2 | A: 0 | A: 1 | A: 0 | A: 1 | A: 1 | A: 1 | A: 1 |
| and after test period | | | | | | | | |
| Pain free exercise | B: 1 | B: 0 | B: 0 | B: 1 | B: 0 | B: 0 | B: 1 | B: 0 |
| in min. before and | A: 39 | A: 60 | A: 50 | A: 55 | A: 40 | A: 44 | A: 48 | A: 43 |
| after test period | | | | | | | | |
| Medication reduced | NA | Yes, after | Yes | Yes, after | NA | Yes, after | NA | NA |
| during test period? | | 5 days | | 1 week | | 2 weeks | | |

TABEL K
Supplement 6(Placebo) & Arthritis Gel

| Testperson | 1 | 2 | 3 | 4 | 5 | 6 | 7 | 8 |
|---|---|---|---|---|---|---|---|---|
| Typical range of | 75-80 | 0-90 | 0-145 | 0-140 | 0-50 | 0-140 | 75-80 | 0-50 |
| motion | degrees | degrees | degrees | degrees | degrees | degrees | degrees | degrees |
| Joint with symptoms | Left | Fingers | Left | Left | Right | Right | Right | Left |
| of arthritis | Wrist | | Elbow | knee | Ankle | Knee | Wrist | Ankle |
| Test period(days) | 91 | 95 | 100 | 116 | 132 | 144 | 160 | 180 |
| minimum 90 days-max | | | | | | | | |
| 180 days | | | | | | | | |
| Joint | B: 50 | B: 70 | B: 130 | B: 120 | B: 48 | B: 130 | B: 60 | B: 38 |
| extension/flexion | A: 20 | A: 45 | A: 65 | A: 48 | A: 25 | A: 50 | A: 13 | A: 15 |
| in degrees ° | | | | | | | | |
| before and after | | | | | | | | |
| test period | | | | | | | | |
| Pain scaled on a | B: 8 | B: 9 | B: 6 | B: 7 | B: 8 | B: 9 | B: 6 | B: 8 |
| 0-10 scale before | A: 2 | A: 2 | A: 2 | A: 1 | A: 1 | A: 1 | A: 0 | A: 0 |
| and after test period | | | | | | | | |
| Pain free exercise | B: 0 | B: 1 | B: + | B: 1 | B: 0 | B: 0 | B: 1 | B: 0 |
| in min. before and | A: 27 | A: 29 | A: 32 | A: 35 | A: 38 | A: 41 | A: 43 | A: 45 |
| after test period | | | | | | | | |
| Medication reduced | NA | Yes | NA | NA | Yes after | NA | Yes | Yes, after |
| during test period? | | | | | 6 weeks | | | 1 week |

### Example 10—Psoriasis Combination Treatment

TABEL L
Supplement 3 (1 capsules a day) & Psoriasis Gel

| Testperson | 1 | 2 | 3 | 4 | 5 | 6 |
|---|---|---|---|---|---|---|
| Psoriasis spot | Scalp - | Hairline | Right Elbow - | Left Hand - | Left Inner | Right Arm - |
| size and position | 3*7 cm | neck - | 3*3 cm | 2*2 cm | Elbow - | 4*6 cm |
| | | 1*12 cm | | | 5*3 cm | |
| Test period(days) | 126 | 118 | 170 | 140 | 125 | 146 |
| minimum 90 days-max | | | | | | |
| 180 days | | | | | | |
| Ich scaled on a 0-10 | B: 8 | B: 7 | B: 9 | B: 8 | B: 6 | B: 8 |
| scale before and | A: 3 | A: 0 | A: 4 | A: 0 | A: 5 | A: 6 |
| after test period | | | | | | |
| Redness & Swelling | B: 5 | B: 6 | B: 7 | B: 7 | B: 7 | B: 7 |
| scaled on a 0-10 | A: 3 | A: 0 | A: 2 | A: 0 | A: 3 | A: 3 |
| scale before and | | | | | | |
| after test period | | | | | | |
| Psoriasis spot | 1*4 cm | gone | 1*1 cm | gone | 2*1 cm | 2*2 cm |
| size in cm after | | | | | | |
| test period | | | | | | |
| Medication reduced | None | None | None | None | None | None |
| during test period? | taking | taking | taking | taking | taking | taking |

TABEL M
Supplement 4 (2 capsules/day) & Psoriasis Gel

| Testperson | 1 | 2 | 3 | 4 | 5 | 6 |
|---|---|---|---|---|---|---|
| Psoriasis spot | Right Hand - | Left Inner | Right Arm - | Scalp Neck - | Back - | Right Elbow - |
| size and position | 3*3 cm | Elbow - | 12*5 cm | 8*4 cm | 15*20 cm | 20*7 cm |
| | | 5*4 cm | | | | |
| Test period(days) | 90 | 110 | 127 | 140 | 180 | 170 |
| minimum 90 days-max | | | | | | |
| 180 days | | | | | | |
| Ich scaled on a 0-10 | B: 9 | B: 8 | B: 7 | B: 8 | B: 9 | B: 6 |
| scale before and | A: 0 | A: 1 | A: 0 | A: 1 | A: 1 | A: 2 |
| after test period | | | | | | |
| Redness & Swelling | B: 7 | B: 6 | B: 8 | B: 5 | B: 6 | B: 7 |
| scaled on a 0-10 | A: 0 | A: 1 | A: 1 | A: 1 | A: 1 | A: 2 |
| scale before and | | | | | | |
| after test period | | | | | | |
| Psoriasis spot | 0 cm | 1*1 cm | Almost gone - | Almost gone | 3*5 cm - | 2*3 cm - red |
| size in cm after | | | slightly red | 1*2 cm | slightly red | |
| test period | | | | slightly red | | |
| Medication reduced | None | None | None | None | Reduced | Reduced |
| during test period? | taking | taking | taking | taking | paracetamol | paracetamol |

TABEL N
Supplement 5 (3 capsules/day) & Psoriasis Gel

| Testperson | 1 | 2 | 3 | 4 | 5 | 6 |
|---|---|---|---|---|---|---|
| Psoriasis spot | Left arm - | Back - | Right Hand | Back - | Left Elbow - | Left Knee - |
| size and position | 2*3 cm | 5*20 cm | 2 spots of | 3*5 cm | 5*5 cm | 6*6 cm |
| | | | 1*1 cm | | | |
| Test period(days) | 90 | 176 | 90 | 90 | 125 | 147 |
| minimum 90 days-max | | | | | | |
| 180 days | | | | | | |
| Ich scaled on a 0-10 | B: 9 | B: 8 | B: 7 | B: 9 | B: 6 | B: 5 |
| scale before and | A: 0 | A: 0 | A: 0 | A: 0 | A: 0 | A: 0 |
| after test period | | | | | | |
| Redness & Swelling | B: 7 | B: 7 | B: 6 | B: 7 | B: 5 | B: 7 |
| scaled on a 0-10 | A: 0 | A: 1 | A: 0 | A: 0 | A: 0 | A: 1 |
| scale before and | | | | | | |
| after test period | | | | | | |
| Psoriasis spot | Gone | Almost gone - | Gone | Gone | Gone | Gone - |
| size in cm after | | slightly red | | | | slightly red |
| test period | | | | | | |
| Medication reduced | None | None | None | None | None | None |
| during test period? | taking | taking | taking | taking | taking | taking |

TABEL O
Supplement 6 (Placebo) & Psoriasis Gel

| Testperson | 1 | 2 | 3 | 4 | 5 | 6 |
|---|---|---|---|---|---|---|
| Psoriasis spot | Upper | Right Knee - | Left knee - | Right Hand - | Hair line - | Lower |
| size and position | Right Arm - | 5*6 cm | 7*6 cm | 4 spots of | 2*8 cm | right arm - |
| | 3*9 cm | | | 1*1 cm | | 8*16 cm |
| Test period(days) | 97 | 136 | 124 | 90 | 90 | 178 |
| minimum 90 days-max | | | | | | |
| 180 days | | | | | | |
| Ich scaled on a 0-10 | B: 7 | B: 5 | B: 6 | B: 8 | B: 8 | B: 9 |
| scale before and | A: 3 | A: 1 | A: 2 | A: 0 | A: 0 | A: 2 |
| after test period | | | | | | |
| Redness & Swelling | B: 8 | B: 6 | B: 6 | B: 7 | B: 6 | B: 9 |
| scaled on a 0-10 | A: 2 | A: 2 | A: 2 | A: 0 | A: 0 | A: 3 |
| scale before and | | | | | | |
| after test period | | | | | | |
| Psoriasis spot | 1*2 cm | 2*2 cm | 3*1 cm | gone | gone | 6*2 cm |
| size in cm after | | | | | | |
| test period | | | | | | |
| Medication reduced | NA | NA | NA | NA | NA | NA |
| during test period? | | | | | | |

### Example 11—Provision of CBD by Alcohol Extraction, Distillation and Crystallization

Crystalline CBD can be provided by methods and techniques known in the art, such as by methods disclosed in U.S. Ser. No. 10/413,845 and/or U.S. Ser. No. 10/414,709.

In short, crystalline CBD can be provided from hemp or cannabis (Cannabis sativa) by a method consisting essentially of:

Extracting hemp or cannabis with a solvent selected from the group consisting of propanol, isopropanol, butanol, pentanol, hexanol, heptanol, and octanol to produce an extract consisting essentially of an extracted hemp or cannabis consisting essentially of tetrahydrocannabinol, a terpene, or cannabidiol;

Evaporating the solvent portion of the extract to generate a substantially solvent-free extract comprising CBD;

Distilling the substantially solvent-free extract to isolate the CBD, and

Crystallizing the distilled, isolated CBD to produce a crystallized, isolated CBD.

Often, the crystallized, isolated CBD is subjected to vacuum drying to remove volatile remnants, in particular the solvent used in crystallizing or re-crystallizing, if needed.

In particular, a method comprising extraction with isopropanol and crystallization by the use of heptane, including one or more optional re-crystallization steps, followed by vacuum drying can provide CBD with crystal structure A, i.e. needle like crystals. Furthermore, such a CBD can be very low in undesired compounds, such as terpenes.

GC chromatography or other analytical methods known in the art can be used to monitor the process such as to ensure a high yield and/or a high purity of the desired product.

Concerning the raw material, hemp comprising e.g. 2-3% CBD is dried and ground before extraction with isopropanol, such as food grade isopropanol.

Guidance for choosing the appropriate reaction based on the boiling points or ranges of the different compounds can e.g. be found here:

www.nwsci.com/customer/docs/SKUDocs/RMR/Technical %20Data Extractions 03.28.18.p df.

Crystal structure A CBD can e.g. be provided from www.enecta.com, and/or following a similar extraction protocol as said manufacturer.

### Example 12—Comparison of Arthritis Gel Compositions Formulated with Different Crystalline CBDs

Two hydroalcoholic arthritis gel compositions are prepared according to Example 2, the only difference being that the crystalline CBD used in the formulation is either of type A (needle-like crystals; FIG. 1) or type B (bunch/cluster-like; FIG. 2).

When testing both arthritis gel compositions, surprisingly and unexpectedly, it is seen that type A CBD is significantly more active than type B CBD!

Type A crystalline CBD is sourced from Enecta, while type B CBD is sourced from Pharma Hemp.

1. A composition, such as a supplement, comprising:
a. 3-O-Acetyl-11-Keto Beta Boswellic Acid (AKBBA);
b. Curcuminoid(s);
c. L-Ascorbic acid (Vitamin C);
d. Cholecalciferol (Vitamin D);
and optionally
e. Piperidine; and/or
f. Hesperidin.

2. Composition according to claim 1, comprising one or more of:
a. 1-15, 1-10, or 1.5-5% by weight 3-O-Acetyl-11-Keto Beta Boswellic Acid (AKBBA);
b. 1-20, 2-15, or 3-10% by weight Curcuminoid(s);
c. 1-20, 1-10, or 2-8% by weight L-Ascorbic acid (Vitamin C);
d. 0.00001-2.0, 0.0001-1.0, 0.0002-0.02% by weight Cholecalciferol (Vitamin D);
e. 0.01-2.0, 0.1-1.5, or 0.5-1.0% by weight Piperidine;
f. 1-20, 1.5-10, or 2.5-3.0% by weight Hesperidin.

3. A composition according to claim 1 or 2, comprising:
a. 1-15, 2-10, 3-5, or around 4% by weight 3-O-Acetyl-11-Keto Beta Boswellic Acid (AKBBA);
b. 2-20, 4-15, 6-10, or around 8% by weight Curcuminoid(s);
c. 1-20, 3-15, 4-8, or around 6% by weight L-Ascorbic acid (Vitamin C);
d. 0.00001-2, 0.0001-1.0, 0.00025-0.001, or around 0.00035% by weight Cholecalciferol (Vitamin D); and/or
e. 0.01-2.0, 0.1-1.5, 0.5-1.0, or around 0.7% by weight Piperidine.

4. A composition according to any one of the preceding claims, comprising:
a. 0.5-15, 1-10, 1.5-2.5, or around 2% by weight 3-O-Acetyl-11-Keto Beta Boswellic Acid (AKBBA); or
b. 1-20, 2-15, 3-6, or around 4% by weight Curcuminoid(s);
c. 1-20, 1.5-10, 2-4, or around 3% by weight L-Ascorbic acid (Vitamin C);
d. 0.00001-2, 0.001-1.0, 0.005-0.02, or around 0.01% by weight Cholecalciferol (Vitamin D); and/or
f. 1-20, 1.5-10, 2.5-3.0, or around 2.8% by weight Hesperidin.

5. Composition according to any one of the preceding claims, formulated for oral intake/consumption, formulated e.g. as a tablet, pill, or capsule, and optionally comprising an excipient, such as Rice Bran extract, e.g. in a concentration of 1-80, 2-70, 3-60, 4-50, 5-30, 8-20, 10-15, or around 12% by weight.

6. Composition according to any one of the preceding claims, wherein said composition is formulated as a capsule comprising 5-30%, 10-25, 15-20, or around 17% by weight capsule shell, said capsule shell optionally comprising a glazing agent, such as Hydroxypropyl Methylcellulose, and/or a colouring agent, such as TiO₂.

7. Composition according to any one of the preceding claims, wherein said composition is formulated as a supplement for daily intake, such as 1-5, e.g. 1, 2 or 3 capsules per day.

8. Composition according to any one of the preceding claims, formulated for oral intake, wherein said tablet, pill or capsule has a weight of around 0.1-2.0 g, 0.4-1.5 g, 0.5-1.0 g, or around 0.72 g.

9. Composition according to any one of the preceding claims, further comprising:
m. Bromelain; and/or
n. Collagen.

10. Composition according to any one of the preceding claims, further comprising:
m. 2-20, 4-15, 7-12, or around 9% by weight Bromelain (1200 GDU/g); and/or
n. 0.5-5, 0.7-2, 1.1-1.8, or around 1.4% by weight Collagen.

11. Composition according to any one of the preceding claims, further comprising one or more of:
q. Rosavin;
r. Zinc;
s. Vitamin B12;
t. Withanolides;
u. Vitamin A;
v. Selene, such as L-Selenomethionine;
w. L-cysteine, such as L-cysteine HCL Mono;
x. L-glutamine;
y. Omega 3 fatty acids; and/or
z. Probiotic bacteria.

12. Composition according to any one of the preceding claims, comprising one or more of:
q. 0.1-5, 0.15-2.0, 0.25-0.8, or around 0.5% by weight Rosavin;
r. 0.25-5, 0.5-2.0, 0.75-1.25, or around 1% by weight Zinc;
s. 0.001-2.0, 0.002-1.0, 0.025-0.01, or around 0.07% by weight Vitamin B12;
t. 0.01-2.0, 0.1-1.5, 0.25-0.75, or around 0.5% by weight Withanolides;
u. 001-2.0, 0.002-1.0, 0.025-0.01, or around 0.07% by weight Vitamin A;
v. 0.0005-2.0, 0.00075-1.0, 0.001-0.005, or around 0.002 or around 0.003% by weight Selene;
y. Omega 3 fatty acids; and/or
z. 0.2-5.0, 0.5-4.0, 1.0-2.0, or around 1.25% by weight Probiotic bacteria (5×10¹⁰ CFU/g).

13. A composition formulated as a supplement comprising:
2-10, 3-5, or around 4% by weight 3-O-Acetyl-11-Keto Beta Boswellic Acid (AKBBA);
4-15, 6-10, or around 8% by weight Curcuminoid(s);
3-15, 4-8, or around 6% by weight L-Ascorbic acid (Vitamin C);
0.0001-1.0, 0.00025-0.001, or around 0.00035% by weight Cholecalciferol (Vitamin D);
0.1-1.5, 0.5-1.0, or around 0.7% by weight Piperidine;
4-15, 7-12, or around 9% by weight Bromelain (1200 GDU/g); and
0.7-2, 1.1-1.8, or around 1.4% by weight Collagen;
and optionally, wherein the supplement is formulated as 1, 2 or 3 capsules of 0.5-1.0, or 0.72 g/day for providing a daily dose.

14. A composition formulated as a supplement, comprising:
0.5-15, 1-10, 1.5-2.5, or around 2% by weight 3-O-Acetyl-11-Keto Beta Boswellic Acid (AKBBA); or
1-20, 2-15, 3-6, or around 4% by weight Curcuminoid(s);
1-20, 1.5-10, 2-4, or around 3% by weight L-Ascorbic acid (Vitamin C);
0.00001-2, 0.001-1.0, 0.005-0.02, or around 0.01% by weight Cholecalciferol (Vitamin D);
1-20, 1.5-10, 2.5-3.0, or around 2.8% by weight Hesperidin.
0.1-5, 0.15-2.0, 0.25-0.8, or around 0.5% by weight Rosavin;
0.25-5, 0.5-2.0, 0.75-1.25, or around 1.0% by weight Zinc;
0.001-2.0, 0.002-1.0, 0.025-0.01, or around 0.07% by weight Vitamin B12;
0.01-2.0, 0.1-1.5, 0.25-0.75, or around 0.5% by weight Withanolides;
001-2.0, 0.002-1.0, 0.025-0.01, or around 0.07% by weight Vitamin A;
0.0005-2.0, 0.00075-1.0, 0.001-0.005, or around 0.003% by weight Selene;
0.2-5.0, 0.5-4.0, 1.0-2.0, or around 1.25% by weight Probiotic bacteria (5×10¹⁰) CFU/g;
and optionally, wherein the supplement is formulated as 1, 2, 3, or 4 capsules of 0.5-1.0, or 0.72 g/day for providing a daily dose.

15. A composition formulated as a supplement, comprising:
0.5-5, 0.7-4.0, 1.0-2.0, or around 1.4% by weight 3-O-Acetyl-11-Keto Beta Boswellic Acid (AKBBA); or
1-20, 2-10, 2-4, or around 2.8% by weight Curcuminoid(s);
1-20, 1.1-10, 1.5-4.0, or around 2% by weight L-Ascorbic acid (Vitamin C);
0.00001-2.0, 0.001-1.0, 0.005-0.02, or around 0.01% by weight Cholecalciferol (Vitamin D);
1-20, 1.2-10, 1.5-2.5, or around 1.85% by weight Hesperidin;
0.001-5.0, 0.15-2.0, 0.2-0.6, or around 0.35% by weight Rosavin;
0.25-5, 0.3.5-2.0, 0.5-1.0, or around 0.7% by weight Zinc;
0.001-2.0, 0.002-1.0, 0.025-0.01, or around 0.05% by weight Vitamin B12;
0.01-2.0, 0.1-1.0, 0.2-0.5, or around 0.3% by weight Withanolides;
001-2.0, 0.002-1.0, 0.025-0.75, or around 0.05% by weight Vitamin A;
0.0005-2.0, 0.00075-1.0, 0.001-0.004, or around 0.002% by weight Selene;
0.1-20, 2-15, 7-12, or around 8.3% by weight L-cysteine and/or L-cysteine HCL Mono;
0.1-20, 2-15, 6-12, or around 9.0% by weigh L-glutamine;
0.1-40, 0.5-20, 1.2-2.5, or around 1.85% by weight Omega 3 fatty acids, such as eicosapentaenoic acid (EPA) and docosahexaenoic acid (DHA)); and/or
0.2-5.0, 0.3-4.0, 0.4-1.3, or around 0.8% by weight Probiotic bacteria (5×10¹⁰ CFU/g).
and optionally, wherein the supplement is formulated as 1, 2, 3, or 4 capsules of 0.5-1.0, or 0.72 g/day for providing a daily dose.

16. A composition according to any one of the preceding claims, wherein one or more of the following components/ingredients are provided such that:
3-O-Acetyl-11-Keto Beta Boswellic Acid (AKBBA) is provided from Boswellia Serrata Resin Extract;
Curcuminoid(s) is are provided from Curcuma Longa Rhizomes Extract;
Vitamin C is provided from Rosehip Fruit Exctract (Rosa canina);
Vitamin D is provided from algae, such as Vita-algae D®;
Piperine is provided from Black Pepper Fruit Extract (Piper nigrum);
Hesperidin is provided from Bitter Orange Peel Extract (Citrus aurantium);
Bromelain is provided from Pineapple (Ananas comosus) (1200 GDU/g);
Collagen is provided from Standardized Chicken Cartilage Powder [25% Total Collagen];
Rosavin is provided from Rhodiola Rosea Root Extract (Rhodiola rosea) [3% Rosavin];
Zinc is provided as Zinc Gluconate;
Vitamin B12 is provided from Cyanocobalamin and/or Methylcobalamin;
Withanolides is provided from Ashwagandha Root Extract (Withania somnifera) [7% Withanolides];
Vitamin A is provided from Retinyl Acetate;
Selene is provided from L-Selenomethionine, such as SeleniumSeLECT®;
L-Cysteine is provided from L-cysteine HCL mono;
Omega 3 fatty acid(s), such as EPA and DHA is/are provided from fish oil, such as microencapsulated fish oil;
Probiotic bacteria are Bacillus coagulans ATCC7050 (5×10¹⁰ CFU/g).

17. Composition according to any one of the preceding claims, wherein said composition does not comprise glucosamine and/or does not comprise glucosamine in a physiologically active or relevant amount.

18. Composition according to any one of the preceding claims, wherein said composition comprises glucosamine in a concentration below 1.0, 0.5, or 0.1% by weight.

19. Composition according to any one of the preceding claims for use as a medicament.

20. Composition according to any one of the preceding claims for use as a medicament and/or in the in the treatment and/or alleviation of symptoms such as pain and/or discomfort in a subject, such as pain or discomfort related to a disease and/or condition, comprising autoimmune-related diseases, stress related conditions, arthritis, rheumatoid arthritis, osteoarthritis, juvenile rheumatoid arthritis, psoriatic arthritis and/or neurological pain, such as pain resulting from sclerosis, psoriasis (e.g. in particular red, dry, itchy, and/or scaly skin), multiple sclerosis., herpes, such as herpes zoster, seborrheic dermatitis, eczema, such as neurodermitis (Lichen simplex chronicus (LSC), atopic dermatitis (AD) aka atopic eczema, Lupus, such as systemic lupus erythematosus (SLE), scleroderma, urticaria, a painful condition associated with one or more hot and/or swollen joints, and/or a skin-, nerve-, joint condition.

21. Composition according to claim 20, wherein the subject is a human, such as one or more of: female, male, senior, adult, adolescent, child, or infant; or an animal, such as a pet, husbandry, and/or mammal.

22. Composition according to any one of the preceding claims for use in the treatment and/or alleviation of symptoms of a skin, nerve and/or joint condition.

23. Composition according to any one of the preceding claims for use in the treatment and/or alleviation of symptoms of an autoimmune condition.

24. Composition according to any one of the preceding claims for use in the treatment and/or alleviation of symptoms related to arthritis.

25. Composition according to any one of the preceding claims for use in the treatment and/or alleviation of symptoms related to psoriasis.

26. Composition according to any one of the preceding claims, wherein said composition is provided orally in combination with a topical composition for use as a medicament and/or in the in the treatment and/or alleviation of symptoms such as pain and/or discomfort in a subject, such as pain or discomfort related to a disease and/or condition, comprising autoimmune-related diseases, stress related conditions, arthritis, rheumatoid arthritis, osteoarthritis, juvenile rheumatoid arthritis, psoriatic arthritis and/or neurological pain, such as pain resulting from sclerosis, psoriasis (e.g. in particular red, dry, itchy, and/or scaly skin), multiple sclerosis., herpes, such as herpes zoster, seborrheic dermatitis, eczema, such as neurodermitis (Lichen simplex chronicus (LSC), atopic dermatitis (AD) aka atopic eczema, Lupus, such as systemic lupus erythematosus (SLE), scleroderma, urticaria, a painful condition associated with one or more hot and/or swollen joints, and/or a skin-, nerve-, joint condition., such as cannabinoid-comprising topical composition.

27. Composition according to claim 26, wherein said cannabinoid is or comprises CBD in a physiological active amount, such as 0.01-5.0, 0.1-2.5, 0.5-2%, or 0.75-1.5%.

28. Composition according to claim 27, wherein said CBD-comprising composition is formulated as cream, ointment, salve, gel, skin-patch, or spray.

29. Composition according to claim 27 or 28, wherein the topical composition is formulated as a hydroalcoholic gel comprising 5-50%, or 10-30% by weight of a low molecular weight alcohol, such as EtOH.

30. Composition according to claim 29, wherein said hydroalcoholic gel is or comprises a composition as disclosed in any one of DK priority application PA 2019 70497, DK priority application PA 2020 70343, and/or WO2021023351, such as disclosed in any one of the claims of said PA 2019 70497, PA 2020 70343, and/or WO2021023351.

31. Composition according to claim 29 or 30, wherein said hydroalcoholic gel comprises one or more of:
i. cannabidiol (CBD) present in an amount of 0.1-20%, such as 0.1-10% or 0.2-5% (by weight);
ii. a skin penetration enhancer, such as a skin penetration enhancer present in an amount of 0.25-5%, or 0.5-2.5% (by weight);
iii. a low molecular weight alcohol, such as ethanol, such as a low molecular weight alcohol present in an amount of 5-50%, or 10-30% (by weight);
iv. one or more thickeners or gelling agents, such as one or more thickeners or gelling agents present in a total amount of 0.2-5%, or 0.4-2% (by weight); and
v. water in a quantity for the composition to a total of 100%; including any combination thereof.

32. Composition according to any one of claims 29-31, wherein said hydroalcoholic gel further comprises: 10-25% (by weight) of sodium chloride, in particular see salt, or more preferred dead sea salt; and optionally, wherein said gel is a gel for use in the treatment of psoriasis; and/or, wherein said gel does not comprise citric acid or citrate.

33. Composition according to any one of claims 29-32, wherein said hydroalcoholic gel further comprises one or more skin care or skin hydrating/moisturizing agents, selected from:
vi. 0.01-1.0% (by weight) extract of aloe barbadensis leaves,
vii. 1-5% (by weight) of panthenol,
viii. 0.1-1-5% (by weight) of retinyl palmitate, and/or
ix. 0.5-5% (by weight) of glycerine,
including any combination(s) thereof.

34. Composition according to any one of claims 29-33, wherein the cannabidiol used in preparation of the hydroalcoholic gel is provided in a crystalline or pure form; and/or wherein the cannabidiol comprises less than 1.5%, 1.0% or 0.5% (by weight) of any one of: Cannabidivarin (CBDV), Cannabidiolic acid (CBDA), Cannabigerol (CBG), Cannabinol (CBN); and/or less than 1.0% of Tetrahydrocannabinol (THC).

35. Composition according to any one of claims 29-34, wherein said hydroalcoholic gel comprises less than 2.0, 1.0%, 0.5, or 0.1% (by weight) oil, such as vegetable and/or mineral oil.

36. Composition according to any one of claims 29-35, wherein said hydroalcoholic gel comprises one or more further components selected from: 0.5-5% (by weight) of menthol; 0.1-2% (by weight) of camphor; and/or 0.5-1.5% (by weight) of eucalyptus oil, including any combination(s) thereof.

37. Composition according to any one of claims 29-36, wherein the skin penetration enhancer is selected from isopropyl myristate, dimethylsulfoxid (DMSO), urea, including any combination(s) thereof.

38. Composition according to any one of claims 29-37, wherein the thickener and/or gelling agent is selected from acrylate cross polymers, hydroxyethyl cellulose, xanthan gum and/or any combinations thereof.

39. Composition according to any one of claims 29-38, wherein said hydroalcoholic gel further comprises one or more pharmaceutically acceptable adjuvants selected from antioxidants, emulsifiers, pH regulating agents, such as acids or bases, buffers, stabilizers, colorants, including any combination(s) thereof.

40. Composition according to any one of claims 26-39, wherein said topical composition is formulated as a cosmetic skin hydrating and/or skin care composition.

41. Composition according to any one of claims 26-40, wherein said topical composition is a composition for use as a medical composition in a local topical application in the treatment or alleviation of symptoms, in particular pain, resulting from arthritis, in particular rheumatoid arthritis, osteoarthritis, juvenile rheumatoid arthritis and/or psoriatic arthritis and/or neurological pain, such as pain resulting from sclerosis, e.g. multiple sclerosis, in a subject, such as a mammal, and/or a subject according to claim 21.

42. Composition according to any one of claims 26-41, wherein said topical composition is a composition for use as a medical composition in local a topical application in the treatment or alleviation of symptoms of psoriasis, in particular of red, dry, itchy, and/or scaly skin, in a subject, such as a subject according to claim 21.

43. Composition according to any one of claims 26-42, wherein said topical composition is applied topically to a local skin area of a subject 1-5, 1-3, or 1-2 times per day.

44. Composition according to any one of claims 26-43, wherein said topical composition is applied topically to a skin area of a subject in an amount of 5-100, or 10-50 mg/cm² per application.

45. Composition according to any one of claims 26-44, wherein said composition does not comprise one or more further cannabinoid(s), such as one or more hallucinogenic and/or non-hallucinogenic cannabinoid, and/or wherein said further cannabinoid(s) is/are present in amount below 10, 5, 2, or 1% by weight in relation to CBD.

46. Composition according to any one of claims 26-45, comprising a further cannabinoid, such as a one or more cannabinoid(s) selected from: THC (tetrahydrocannabinol), THCA (tetrahydrocannabinolic acid), CBDA (cannabidiolic acid), CBN (cannabinol), CBG (cannabigerol), CBC (cannabichromene), CBL (cannabicyclol), CBV (cannabivarin), THCC (tetrahydrocannabiorcol), THCV (tetrahydrocannabivarin), THCP (tetrahydrocannabiphorol), CBDV (cannabidivarin), CBCV (cannabichromevarin), CBGV (cannabigerovarin), CBGM (cannabigerol monomethyl ether), CBE (cannabielsoin), CBT (cannabicitran), including one or more cannabinoids of the following types: CBG-type, CBC-type, CBD-type other than CBD, THC-type, CBN-type, CBE-type, iso-THC-type, CBL-type, CBT-type, including any combination(s) thereof.

47. Composition according to claim 46, wherein said further cannabinoid is provided in a ratio CBD:further cannabinoid of >10:1, 10:1-5:1, 5:1-2:1, 2:1-1:1, 1:1-1:2, 1:2-1:5, or <1:5.

48. Composition according to any one of claims 29-47, wherein said hydroalcoholic gel is formulated in particular for treatment of arthritis-related conditions, said gel comprising:
0.5-2.0% (by weight) or more preferred ˜1.0% (by weight) of cannabidiol;
10-30% (by weight) of ethanol or more preferred ˜25% (by weight) of ethanol, in particular 96% ethanol, such as 96% denatured ethanol;
0.5-5% (by weight) or more preferred ˜2% (by weight) menthol;
0.1-2% (by weight) or more preferred 0.55% (by weight) of camphor;
0.5-1.5% (by weight) or more preferred ˜0.9% skin penetration enhancer, such as isopropyl myristate;
0.4-2% (by weight) or more preferred ˜1% thickeners and/or gelling agents, such as acrylate cross-polymer;
0.05-0.5% (by weight) or more preferred ˜0.2% phytic acid (e.g. of 50% purity); and
water in a quantity for the composition to a total of 100% (by weight), such as 50-80, or around ˜68% (by weight), said water being preferably water of drinking water quality.

49. Composition according to any one of claims 29-47, wherein said hydroalcoholic gel is formulated in particular for treatment of psoriasis-related conditions, said gel comprising:
0.5-2.0% (by weight) or more preferred ˜1.0% (by weight) cannabidiol;
0.3-1.5% (by weight) or more preferred ˜0.9% (by weight) skin penetration enhancer, in particular isopropyl myristate;
0.1-0.5% (by weight) or more preferred ˜0.2% (by weight) glycerine;
10-30% (by weight) or more preferred ˜15% (by weight) ethanol, in particular 96% ethanol, such as 96% denatured ethanol;
0.5-2.5% (by weight) or more preferred ˜1.5% (by weight) one or more thickeners or gelling agents, in particular hydroxyethyl cellulose;
10-20% (by weight) or more preferred ˜15.0% (by weight) sodium chloride, in particular sea salt, or more preferred Dead Sea salt;
% (by weight) or more preferred ˜0.05% (by weight) extract of aloe barba-densis leaves;
1.5-4.0% (by weight) or more preferred ˜2.75% (by weight) panthenol;
0.1-0.5% (by weight) or more preferred ˜0.3% (by weight) retinyl palmitate;
0.1-0.3% (by weight) or more preferred ˜0.2% (by weight) glycerine;
0.01-0.03% (by weight) or more preferred ˜0.02% (by weight) of an organic acid or salt thereof, e.g. lactic acid and/or acetic acid; and
water in a quantity for the composition to a total of 100% (by weight), such as 50-80% (by weight), more preferred ˜61% (by weight), said water being preferably water of drinking water quality.

50. Composition according to any one of claims 27-49, wherein the CBD used in the provision of the topical composition is crystalline.

51. Composition according to claim 50, wherein the CBD crystals used in the formulation of the topical composition are needle-like crystals, such as crystals shown in FIG. 1.

52. Composition according to claim 50 or 51, wherein the CBD crystals used in the formulation of the topical composition are not cluster- or bunch-shaped, such as crystals similar to crystals shown in FIG. 2.

53. Composition according to any one of claims 50-52, wherein the CBD crystals are not provided by an extraction method comprising critical C02 extraction.

54. Composition according to any one of claims 50-53, wherein the CBD crystals are provided by a method comprising extraction with a C₃-C₄ alcohol, such as isopropanol, and one or more crystallisations steps with a C₆-C₈ alcohol, such as heptane.

55. Composition according to any one of claims 50-54, wherein the CBD crystals are provided by a method comprising critical CO₂ extraction and one or more crystallisations steps with a C₆-C₈ alkane, such as heptane.

56. Composition according to claim 54 and 55, wherein the C₃-C₄ alcohol is isopropanol, and the C₆-C₈ alkane is heptane.

57. Composition according to any one of claims 50-56, wherein the crystalline CBD does not comprise significant amounts of terpenes, such as less than 0.1, less than 0.05, less than 0.02, less than 0.01, less than 0.005, less than 0.002, less than 0.001%/terpenes by weight.

58. Composition according to any one of claims 50-57, wherein the crystalline CBD does not comprise significant amounts of terpenoids, such as less than 0.1, less than 0.05, less than 0.02, less than 0.01, less than 0.005, less than 0.002, less than 0.001%/terpenoids by weight.

59. CBD-comprising composition according to any one of claims 50-58, wherein the CBD possesses a conformation of CBD capable of forming needle-like crystals, such as crystals shown in FIG. 1.

60. Method of treatment and/or alleviation of symptoms such as pain and/or discomfort in a subject, such as a subject according to claim 21, wherein said pain and/or discomfort relates to a disease and/or condition, comprising autoimmune-related diseases, stress related conditions, such as one or more of: arthritis, rheumatoid arthritis, osteoarthritis, juvenile rheumatoid arthritis, psoriatic arthritis and/or neurological pain, such as pain resulting from sclerosis, psoriasis (e.g. in particular red, dry, itchy, and/or scaly skin), multiple sclerosis (MS), herpes, such as herpes zoster, seborrheic dermatitis, eczema, neurodermitis, Lichen simplex chronicus (LSC), atopic dermatitis (AD) aka atopic eczema, Lupus, such as systemic lupus erythematosus (SLE), scleroderma, urticaria, a painful condition associated with one or more hot and/or swollen joints; and/or a skin-, nerve-, joint condition; wherein said method comprises the use of a composition, such as a supplement according to any one of the preceding claims.

61. Method according to claim 60, wherein said treatment and/or alleviation of symptoms are related to arthritis or psoriasis.

62. Method according to claim 61, wherein said supplement is a composition according to any one of claims 1-3, 5-10, 13, 16-18, and said treatment is related to arthritis.

63. Method according to claim 60 or 61, wherein said supplement is a composition according to any one of claims 1-2, 4-8, 11-12, 14-18, and said treatment is related to psoriasis.

64. Method according to any one of claims 60-63, further comprising the use of a cannabinoid-comprising topical composition.

65. Method according to claim 64, comprising the use of a cannabinoid-comprising topical composition, according to any one of claims 27-59.

66. Method according to any one of claims 60-65, further comprising the use of a CBD-comprising hydroalcoholic gel, such as a hydroalcoholic gel according to any one of claims 29-59.

67. Method according to claim 62, comprising the use of a CBD-comprising hydroalcoholic gel according to claim 48.

68. Method according to claim 63, comprising the use of a CBD-comprising hydroalcoholic gel according to claim 49.

69. A receptacle comprising a composition according to any one of the preceding claims.

70. Receptacle according to claim 69, wherein said receptacle provides protection from visible and/or UV-light.

71. Receptacle according to claim 69 or 70, comprising a supplement according to any one of claims 1-59.

72. Receptacle according to claim 69 or 70, comprising a topical composition, such as a CBD alcoholic gel, according to any one of claims 26-59.

73. A kit comprising a receptacle according to any one of claims 69-72, an instruction for use, and optionally a packaging.

74. Kit according to claim 73, comprising at least one receptacle according to claim 71, and at least one receptacle according to claim 72.

75. A CBD-comprising composition according to any one of claims 51-59.
